(19) United States
(12) Patent Application Publication
(10) Pub. No.: US 2011/0015295 A1
(43) Pub. Date: Jan. 20, 2011

(54) ADDITIVE MIXTURES FOR AGRICULTURAL ARTICLES

(76) Inventors: STEFANO GARDI, Bologna (IT); Piero Piccinelli, Sasso Marconi (BO) (IT); Massimiliano Sala, Castelnuovo Rangone (MO) (IT); Wiebke Wunderlich-Wippert, Lorrach (DE); Michela Bonora, Bologna (IT)

Correspondence Address: BASF Corporation;Patent Department, 500 White Plains Road, P.O. Box 2005, Tarrytown, NY, 10591, US

(21) Appl. No.: 12/890,837

(22) Filed: Sep. 27, 2010

Related U.S. Application Data
(63) Continuation of application No. 11/887,499, filed on Sep. 28, 2007.

(30) Foreign Application Priority Data
Apr. 5, 2005 (EP) 05102659.9

(51) Int. Cl. C07D 251/46; C07C 50/18; C08J 3/28; C08L 3/02; C08K 5/08; C08K 3/04

(52) U.S. Cl. 522/48; 544/187; 552/298; 106/209.1; 106/215.1; 106/499; 106/474

(57) ABSTRACT

Disclosed are 2,6-bis[octadecyloxy]anthraquinone, 1-(4,6-bis[dibutylamino]-[1,3,5]-triazin-2-ylamino)-anthraquinone, 2-(4,6-bis[dibutylamino]-[1,3,5]-triazin-2-ylaminoyanthraquinone, 2,6-bis[4,6-bis[dibutylamino]-[1,3,5]-triazin-2-yloxy]-anthraquinone, 2-(2-ethyl-hexyloxy)-1-octadecyloxy-anthraquinone, 1,2-bis[octadecyloxy]anthraquinone, 1,2-bis[2-ethyl-hexyloxy]anthraquinone, 1,2-bis[octyloxy]anthraquinone, 1,5-bis[2-ethyl-hexyloxy]anthraquinone, 9,10-dioxo-9,10-dihydro-anthracene-2-sulfonic acid dodecylamide, 2,6-bis[stearoyloxy]anthraquinone and bis[9,10-dioxo-9,10-dihydro-anthracene-2-sulfonic acid]-1′,12′-dodecandiyldiamide.

The anthraquinones are suitable additives for thermoplastic polymers or waxes and act as photosensitizers towards their degradation.

This application is a continuation of U.S. application Ser. No. 11/887,499, pending, which is a national stage application of PCT/EP 2006/061048, filed Mar. 27, 2006, the contents of which are incorporated by reference.

The present invention relates in particular to an agricultural article made of a composition containing (a) an organic polymer and (b) an additive mixture comprising a certain photosensitizer, an organic salt of a transition metal and optionally an inorganic peroxide or inorganic superoxide; to the use of the additive mixture for controlling the weathering resistance and the degradation of the agricultural article; as well as to several additive mixtures per se and to novel anthraquinones.

Plastic articles find widespread applications in everyday life because of their durability in use and cost effectiveness. With proper stabilization, most commercial plastics are made to last for years.

In recent years however, environmental concern has lead to the development of so called biodegradable materials, of diverse origin and nature, which will maintain their function and integrity during service life, but disintegrate after use into carbon dioxide and water, either triggered by chemical means or by microorganisms. One problem however is establishing a suitable equilibrium between biodegradability and integrity during service life.

The degradation of polymers is for example described in U.S. Pat. No. 4,038,227; U.S. Pat. No. 5,258,422; U.S. Pat. No. 5,854,304; GB-A-1,434,641; EP-A-546,530; EP-A-814,184; WO-A-03/50,178 and in Biomaterials 21 (2000)1235-1246 by G.L.Y. Woo et al. Oxygen scavenging compositions are for example described in U.S. Pat. No. 5,776,361; the stabilization of polymeric organic compounds is for example described in U.S. Pat. No. 2,507,142; particular olefin block copolymers are for example described in EP-A-1,275,670; and an easily degradable and removable filter cake is for example described in U.S. Pat. No. 5,783,527.

The present invention especially relates to

an agricultural article made of a composition containing
(a) an organic polymer, and
(b) an additive mixture comprising the components
(b-0) a photosensitizer,
(b-I) an organic salt of a transition metal, and optionally
(b-II) an inorganic peroxide or inorganic superoxide.

Component (b-I) is preferably a metal salt of a fatty acid with a carbon number ranging from C₂ to C₃₆, in particular from C₁₂ to C₃₆. Particularly preferred examples are metal carboxylates of palmitic acid (C₁₆), stearic acid (C₁₈), oleic acid (C₁₈), linoleic acid (C₁₈) and linolenic acid (C₁₈). Further examples of component (b-I) are aromatic acids, e.g. benzoic acid. Component (b-I) as C₂-C₃₆carboxylate of Fe, Ce, Co, Mn, Cu or V is of particular interest.

A further preferred embodiment of the present invention relates to component (b-I) as a C₁₂-C₂₀alkanoate of Mn, or a C₁₂-C₂₀alkenoate of Mn.

Component (b-II) is an inorganic peroxide or an inorganic superoxide, preferably of an alkali metal or an alkaline earth metal, or of a transition metal. Suitable examples of component (b-II) as a peroxide are magnesium peroxide (MgO₂), calcium peroxide (CaO₂), strontium peroxide (SrO₂), barium peroxide (BaO₂), lithium peroxide (Li₂O₂), sodium peroxide (Na₂O₂), potassium peroxide (K₂O₂), zinc peroxide (ZnO₂), silver peroxide (Ag₂O₂), copper peroxide or iron peroxide. Suitable examples of component (b-II) as a superoxide are lithium superoxide (LiO₂), sodium superoxide (NaO₂), potassium superoxide (KO₂), rubidium superoxide (RbO₂) and cesium superoxide (CsO₂). Preferred are lithium peroxide, sodium peroxide, magnesium peroxide, calcium peroxide, barium peroxide, zinc peroxide and potassium superoxide. Particularly preferred are sodium peroxide, magnesium peroxide, calcium peroxide and zinc peroxide. A comprehensive summary of inorganic peroxides or superoxides, which are useful for the present invention, is given in Kirk-Othmer, Encyclopedia of Chemical Technology, John Wiley & Sons, 4. ed. (1994), Vol. 18, pages 202-229, which is incorporated by reference herein. Blends of the abovementioned peroxides and/or superoxides can also be used.

Component (b-II) is preferably an inorganic peroxide of an alkali metal, an alkaline earth metal or a transition metal, or an inorganic superoxide of an alkali metal, an alkaline earth metal or a transition metal.

Further preferred examples of component (b-II) are magnesium peroxide, calcium peroxide, strontium peroxide, barium peroxide, lithium peroxide, sodium peroxide, potassium peroxide, zinc peroxide, silver peroxide, copper peroxide, iron peroxide, lithium superoxide, sodium superoxide, potassium superoxide, rubidium superoxide and cesium superoxide; in particular sodium peroxide, magnesium peroxide, calcium peroxide and zinc peroxide; especially calcium peroxide.

Component (b-II) is conveniently a compound which—when in contact with humidity—reacts with water in order to liberate an active auxiliary prodegradant species, and which is thermoprocessable in a temperature range between 100° and 300° C.

Component (b-0) is preferably a polyterpene resin or an anthraquinone derivative.

The polyterpene resins used according to the present invention may be of natural or synthetic origin. They are either commercially available or can be prepared according to known methods.

The polyterpene resins are for example based on acyclic terpenes or cyclic terpenes, e.g. monocyclic terpenes or bicyclic terpenes. Polyterpenes based on terpene hydrocarbons are preferred.

Examples of acyclic terpenes are

terpene hydrocarbons, e.g.
myrcene, ocimene and beta-farnesene;
terpene alcohols, e.g.
dihydromyrcenol (2,6-dimethyl-7-octen-2-ol), geraniol (3,7-dimethyl-trans-2,6-octadien-1-ol), nerol (3,7-dimethyl-cis-2,6-octadien-1-ol), linalool (3,7-dimethyl-1,6-octadien-3-ol), myrcenol (2-methyl-6-methylene-7-octen-2-ol), lavandulol, citronellol (3,7-dimethyl-6-octen-1-ol), trans-trans-farnesol (3,7,11-trimethyl-2,6,10-dodecatrien-1-ol) and trans-nerolidol (3,7,11-trimethyl-1,6,10-dodecatrien-3-ol);
terpene aldehydes and acetals, e.g.
citral (3,7-dimethyl-2,6-octadien-1-al), citral diethyl acetal (3,7-dimethyl-2,6-octadien-1-aldiethyl acetal), citronellal (3,7-dimethyl-6-octen-1-al), citronellyloxyacetaldehyde and 2,6,10-trimethyl-9-undecenal;
terpene ketones, e.g.
tagetone, solanone and geranylacetone (6,10-dimethyl-5,9-undecadien-2-one);
terpene acids and esters, e.g.
cis-geranic acid, citronellic acid, geranyl esters (including geranyl formate, geranyl acetate, geranyl propionate, geranyl isobutyrate and geranyl isovalerate), neryl esters (including neryl acetate), linalyl esters (including linalyl formate, linalyl acetate, linalyl propionate, linalyl butyrate and linalyl isobutyrate), lavandulyl esters (including lavandulyl acetate), citronellyl esters (including citronellyl formate, citronellyl acetate, cintronellyl propionate, citronellyl isobutyrate, citronellyl isovalerate and citronellyl tiglate); and
nitrogen containing unsaturated terpene derivatives, e.g.
cis-geranic acid nitrile and citronellic acid nitrile.

Examples of cyclic terpenes are

cyclic terpene hydrocarbons, e.g.
limonene (1,8-p-methadiene), alpha-terpinene, gamma-terpinene (1,4-p-menthadiene), terpinolene, alpha-phellandrene (1,5-p-menthadiene), beta-phellandrene, alpha-pinene (2-pinene), beta-pinene (2(10)-pinene), camphene, 3-carene, caryophyllene, (+)-valencene, thujopsene, alpha-cedrene, beta-cedrene and longifolene;
cyclic terpene alcohols and ethers, e.g.
(+)-neoiso-isopulegol, isopulegol (8-p-menten-3-ol), alpha-terpineol (1-p-menten-8-ol), beta-terpineol, gamma-terpineol, delta-terpineol and 1-terpinen-4-ol (1-p-menten-4-ol);
cyclic terpene aldehydes and ketones, e.g.
carvone (1,8-p-mantadien-6-one), alpha-ionone (C₁₃H₂₀O), beta-ionone (C₁₃H₂₀O), gamma-ionone (C₁₃H₂₀O), irone (alpha-, beta-, gamma-) (C₁₄H₂₂O), n-methylionone (alpha-, beta-, gamma-) (C₁₄H₂₂O), isomethylionone (alpha-, beta-, gamma-) (C₁₄H₂₂O), allylionone (C₁₆H₂₄O), pseudoionone, n-methylpseudoionone, isomethylpseudoionone, damascones (1-(2,6,6-trimethylcycohexenyl)-2-buten-1-ones; including beta-damascenone (1-(2,6,6-trimethyl-1,3-cyclohadienyl)-2-buten-1-one)), nootkatone (5,6-dimethyl-8-isopropenylbicyclo[4.4.0]-1-decen-3-one) and cedryl methyl ketone (C₁₇H₂₆O); and
cyclic terpene esters, e.g.
alpha-terpinyl acetate (1-p-menthen-8-ylacetate), nopyl acetate ((−)-2-(6,6-dimethylbicyclo[3.1.1]hept-2-en-2-yl) ethyl acetate) and khusymil acetate.

Further suitable terpene derivatives can be found in Kirk-Othmer, Encyclopedia of Chemical Technology, John Wiley & Sons, 4. ed. (1994), Vol. 23, p. 833-882.

Preferred examples of terpenes which can serve as the basis for the polyterpenes are tricyclene, alpha-pinene, alpha-fenchene, camphene, beta-pinene, myrcene, cis-pinane, cis/trans-p-8-menthene, trans-2-p-menthene, p-3-menthene, trans-p-menthane, 3-carene, cis-p-menthane, 1,4-cineole, 1,8-cineole, alpha-terpinene, p-1-menthene, p-4(8)-menthene, limonene, p-cymene, gamma-terpinene, p-3,8-menthadiene, p-2,4(8)-menthadiene and terpinolene.

Further examples of the photosensitizers of component (b-0) are cycloaliphatic compounds structurally related to terpenes such as the following

alcohols, e.g.
5-(2,2,3-trimethyl-3-cyclopenten-1-yl)-3-methylpentan-2-ol;
aldehydes, e.g.
2,4-dimethyl-3-cyclohexene carboxaldehyde, 4-(4-methyl-3-penten-1-yl)-3-cyclohexene carboxaldehyde and 4-(4-hydroxy-4-methylpentyl)-3-cyclohexene carboxaldehyde;
ketones, e.g.
civetone, dihydrojasmone (3-methyl-2-pentyl-2-cyclopenten-1-one), cis-jasmone (3-methyl-2-(2-cis-penten-1-yl)-2-cyclopenten-1-one), 5-cyclohexadecen-1-one, 2,3,8,8-tetramethyl-1,2,3,4,5,6,7,8-octahydro-2-napthalenyl methyl ketone and 3-methyl-2-cyclopenten-2-ol-1-one; and
esters, e.g.
4,7-methano-3a,4,5,6,7,7a-hexahydro-5-(or 6)-indenyl acetate, allyl 3-cyclohexylpropionate, methyl dihydrojasmonate methyl (3-oxo-2-pentylcyclopentyl)acetate.

The polyterpenes used in the present invention may also be derived from the copolymerisation of the aforementioned terpenes with other unsaturated organic compounds.

Other examples of the photosensitizers of component (b-0) are the unsaturated coal-tar by-product polymers such as cumarone-indene resins, rosin and the like.

Present component (b-0) is preferably a polyterpene resin selected from the group consisting of poly-alpha-pinene, poly-beta-pinene, polylimonene or a copolymer of alpha-pinene, a copolymer of beta-pinene or a copolymer of limonene. Poly-beta-pinene is particularly preferred.

Terpene-based hydrocarbon resins are typically based on products such as alpha-pinene, beta-pinene and d-limonene, which are obtained from the wood and citrus industry, respectively. Terpene-based resins have been available since the mid-1930s (Kirk-Othmer, Encyclopedia of Chemical Technology, John Wiley & Sons, 4. ed. (1994), Vol. 13, p. 717-718). Polymerization of monoterpenes is most commonly accomplished by carbocationic polymerization utilizing Friedel-Crafts-type catalyst systems, such as aluminum chloride (Kirk-Othmer, Encyclopedia of Chemical Technology, John Wiley & Sons, 4. ed. (1994), Vol. 1, p. 459).

In general, the polyterpenes of the present invention have more than one terpene unit. They have preferably a molecular weight of about 400 g/mol to about 1400 g/mol.

The anthraquinone derivative used according to the present invention preferably corresponds to the formula (A) or (B)

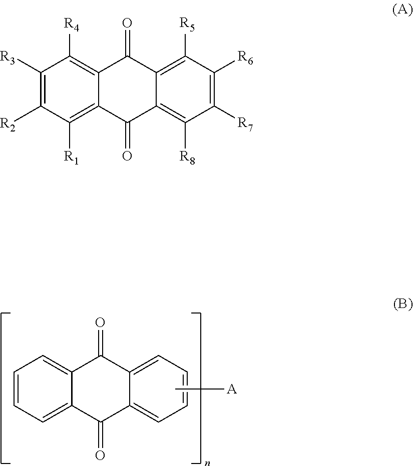

wherein R₁, R₂, R₃, R₄, R₅, R₆, R₇ and R₈ independently of one another are hydrogen,
C₁-C₂₀alkyl, preferably C₄-C₂₀alkyl, in particular C₈-C₂₀alkyl;
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉-phenylalkyl,
C₇-C₉phenylalkyl which is substituted on the phenyl by 1, 2 or 3 radicals selected from the group consisting of C₁-C₁₀alkyl, C₁-C₁₀alkyloxy and hydroxy;
an unsubstituted or substituted 5 to 6 ring-membered heterocyclic group,
a polyoxyalkylene residue; or a group

### —O—X₁,

### —C(O)—X₂,

### —O—C(O)—X₃,

### —C(O)—O—X₄,

### —N(X₅)(X₆),

### —S—X₇,

### —SO₂—O—X₈, or

### —SO₂—N(X₉)(X₁₀)

X₁, X₂, X₃, X₄, X₅, X₆, X₇, X₈, X₉ and X₁₀ independently of one another are hydrogen,
C₁-C₂₀alkyl, preferably C₄-C₂₀alkyl, in particular C₈-C₂₀alkyl;
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkyloxy and hydroxy;
an unsubstituted or substituted 5 to 6 ring-membered heterocyclic group, or
a polyoxyalkylene residue;
with the proviso that at least one radical of the group consisting of R₁, R₂, R₃, R₄, R₅, R₆, R₇ and R₈ is different from hydrogen;
n is 2 or 3,
when n is 2, A is a group —Y₁—Z₁—Y₂— or a group —SO₂—N(G₀)-Z₁—N(G₁)-SO₂—,
Y₁ and Y₂ independently of one another are >N-G₂, —O— or —S—,
Z₁ is C₂-C₁₂alkylene, C₂-C₁₆alkylene interrupted by oxygen, sulphur or >N-G₃;
C₂-C₁₂alkenylene, C₂-C₁₂alkynylene, C₅-C₁₂cycloalkylene,
C₅-C₁₂cycloalkylene-(C₁-C₄alkylene)-C₅-C₁₂cycloalkylene,
C₁-C₄alkylene-(C₅-C₁₂cycloalkylene)-C₁-C₄alkylene, phenylene,
phenylene-(C₁-C₄alkylene)-phenylene or C₁-C₄alkylene-phenylene-C₁-C₄alkylene or a group of the formula

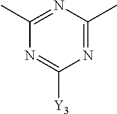

### Y₃ is —O-G₄, —S-G₅ or —N(G₆)(G₇),

G₀, G₁, G₂, G₃, G₄, G₅, G₆ and G₇ independently of one another are
hydrogen,
C₁-C₂₀alkyl,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkoxy and hydroxy;
an unsubstituted or substituted 5 to 6 ring-membered heterocyclic group, or
a polyoxyalkylene residue;
when n is 3, A is a group —Y₄—Z₂(Y₅—)(Y₆—),
Y₄, Y₅ and Y₆ independently of one another are —N(G₈)-, —O—, —S—, or —N(G₉)-SO₂—,
G₈ and G₉ independently of one another are
hydrogen,
C₁-C₂₀alkyl,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkoxy and hydroxy;
an unsubstituted or substituted 5 to 6 ring-membered heterocyclic group, or
a polyoxyalkylene residue, and
Z₂ is C₅-C₂₅alkantriyl or 2,4,6-triazintriyl.

Examples of C₁-C₂₀alkyl are methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methyl-hexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methyl-undecyl, dodecyl, 1,1,3,3,5,5-hexamethylhexyl, tridecyl, tetradecyl, pentadecyl, hexadecyl, heptadecyl, octadecyl and eicosyl. C₄-C₂₀alkyl, in particular C₈-C₂₀alkyl, is preferred.

Examples of C₁-C₂₀alkyl substituted by C₁-C₁₀alkylamino, di(C₁-C₁₀alkyl)amino, C₁-C₁₀alkyloxy or hydroxy are 3-methylaminopropyl, 2-dimethylaminoethyl, 2-diethylaminoethyl, 3-dimethylaminopropyl, 3-diethylaminopropyl, 2-methoxyethyl, 2-ethoxyethyl, 2-methoxypropyl, 2-ethoxypropyl, 3-isopropoxypropyl and hydroxyethyl.

Examples of C₃-C₂₀alkenyl are allyl, 2-methallyl, butenyl, pentenyl, hexenyl and oleyl. The carbon atom in position 1 is preferably saturated. Particularly preferred examples are allyl and oleyl.

Examples of C₅-C₁₂cycloalkyl are cyclopentyl, cyclohexyl, cycloheptyl, cyclooctyl and cyclododecyl.

A preferred example of C₅-C₁₂cycloalkyl substituted by 1, 2 or 3 C₁-C₁₀alkyl is 2-methylcyclohexyl.

An example of C₅-C₉cycloalkenyl is cyclohexenyl.

An example of C₅-C₉cycloalkenyl substituted by 1, 2 or 3 C₁-C₁₀alkyl is methylcyclohexenyl.

Examples of phenyl substituted by 1, 2 or 3 radicals selected from the group consisting of C₁-C₁₀alkyl, C₁-C₁₀alkyloxy and hydroxy are 4-methylphenyl, 2-ethylphenyl, 4-ethylphenyl, 4-isopropylphenyl, 4-tert-butylphenyl, 4-sec-butylphenyl, 4-isobutylphenyl, 3,5-dimethylphenyl, 3,4-dimethylphenyl, 2,4-dimethylphenyl, 2,6-diethylphenyl, 2-ethyl-6-methylphenyl, 2,6-diisopropylphenyl, 4-methoxyphenyl, 4-ethoxyphenyl and 4-hydroxyphenyl.

Examples of C₇-C₉phenylalkyl are benzyl and 2-phenylethyl.

Examples of C₇-C₉phenylalkyl which is substituted on the phenyl by 1, 2 or 3 radicals selected from the group consisting of C₁-C₁₀alkyl, C₁-C₁₀alkyloxy and hydroxy are methylbenzyl, dimethylbenzyl, trimethylbenzyl, tert-butylbenzyl, methoxybenzyl and 3,5-di-tert-butyl-4-hydroxybenzyl.

Examples of an unsubstituted or substituted 5 to 6 ring-membered heterocyclic group are 1-pyrrolidyl, piperidino, morpholino, 1-piperazinyl, 4-methyl-1-piperazinyl, 1-hexahydroazepinyl, 5,5,7-trimethyl-1-homopiperazinyl or 4,5,5,7-tetramethyl-1-homopiperazinyl as well as the group

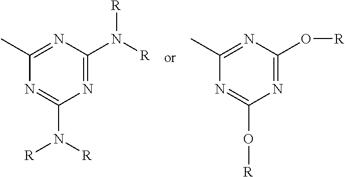

wherein the radicals R independently of one another are hydrogen or C₁-C₂₀alkyl, in particular —C₄H₉.

Examples of a polyoxyalkylene residue correspond to the formula

—O—(CHZ₃—(CH₂)ᵣ—O)_{q}—CHZ₄—(CH₂)ᵣ—OZ₅

wherein Z₃ and Z₄ independently of one another are hydrogen or C₁-Cₐalkyl, Z₅ is C₁-C₂₀alkyl, r is 1, 2, 3 or 4 and q is a number from 1 to 100.

Examples of C₂-C₁₂alkylene are ethylene, propylene, trimethylene, tetramethylene, pentamethylene, hexamethylene, octamethylene, decamethylene and dodecamethylene.

Examples of C₂-C₁₆alkylene interrupted by oxygen, sulphur or >N-G₃ are 3-oxapentane-1,5-diyl, 4-oxaheptane-1,7-diyl, 3,6-dioxaoctane-1,8-diyl, 4,7-dioxadecane-1,10-diyl, 4,9-dioxadodecane-1,12-diyl, 3,6,9-trioxaundecane-1,11-diyl, 4,7,10-trioxamidecane-1,13-diyl, 3-thiapentane-1,5-diyl, 4-thiaheptane-1,7-diyl, 3,6-dithiaoctane-1,8-diyl, 4,7-dithiadecane-1,10-diyl, 4,9-dithiadodecane-1,12-diyl, 3,6,9-trithiaundecane-1,11-diyl, 4,7,10-trithiamidecane-1,13-diyl and —CH₂CH₂CH₂—N(G₃)—CH₂CH₂—N(G₃)—CH₂CH₂CH₂—, in particular —CH₂CH₂CH₂—N(CH₃)—CH₂CH₂—N(CH₃)—CH₂CH₂CH₂—.

An example of C₂-C₁₂alkenylene is 3-hexenylene.

An example of C₂-C₁₂alkynylene is —CH₂CH₂—C≡C—CH₂CH₂—. C₆-C₁₂alkynylene is preferred.

An example of C₅-C₁₂cycloalkylene is cyclohexylene.

Examples of C₅-C₁₂cycloalkylene-(C₁-C₄alkylene)-C₅-C₁₂cycloalkylene are methylenedicyclohexylene and isopropylidenedicyclohexylene.

An example of C₁-C₄alkylene-(C₅-C₁₂cycloalkylene)-C₁-C₄alkylene is cyclohexylenedimethylene.

An example of phenylene-(C₁-C₄alkylene)-phenylene is methylenediphenylene.

An example of C₁-C₄alkylene-phenylene-C₁-C₄alkylene is phenylenedimethylene.

C₅-C₂₅alkantriyl may be for example a group H₃C—C(CH₂—)₃.

An anthraquinone derivative of the formula (A)

wherein 7 radicals of the group consisting of R₁, R₂, R₃, R₄, R₅, R₆, R₇ and R₈ are hydrogen and one radical of this group is different from hydrogen is preferred.

An anthraquinone derivative of the formula (A)

wherein 6 radicals of the group consisting of R₁, R₂, R₃, R₄, R₅, R₆, R₇ and R₈ are hydrogen and two radicals of this group are different from hydrogen is further preferred.

An anthraquinone derivative of the formula (A)

wherein R₁, R₂, R₃, R₄, R₆, R₇ and R₈ are hydrogen and R₅ is different from hydrogen, or wherein R₁, R₂, R₃, R₄, R₅, R₇ and R₅ are hydrogen and R₆ is different from hydrogen, or wherein R₁, R₃, R₄, R₅, R₇ and R₈ are hydrogen and R₂ and R₆ are different from hydrogen, or wherein R₁, R₂, R₃, R₄, R₇ and R₈ are hydrogen and R₅ and R₆ are different from hydrogen, or wherein R₂, R₃, R₄, R₆, R₇ and R₈ are hydrogen and R₁ and R₅ are different from hydrogen is particularly preferred.

An anthraquinone derivative of the formula (A)

wherein R₃, R₄, R₇ and R₈ are hydrogen is also particularly preferred.

An anthraquinone derivative of the formula (A)

wherein R₁, R₂, R₃, R₄, R₅, R₆, R₇ and R₈ independently of one another are
hydrogen,
C₈-C₂₀alkyl, or a group

### —O—X₁,

### —O—C(O)—X₃,

### —N(X₅)(X₆), or

### —SO₂—N(X₉)(X₁₀),

X₁ is hydrogen, C₈-C₂₀alkyl, or
an unsubstituted or substituted 5 to 6 ring-membered heterocyclic group;
X₁, X₃, X₅, X₆, X₉ and X₁₀ independently of one another are
hydrogen,
C₁-C₂₀alkyl, or
an unsubstituted or substituted 5 to 6 ring-membered heterocyclic group, in particular

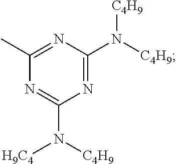

with the proviso, that one or two radicals of the group consisting of R₁, R₂, R₃, R₄, R₅, R₆, R₇ and R₈ are different from hydrogen and the remaining radicals of this group are hydrogen, is of interest.

An anthraquinone derivative of the formula (B-1)

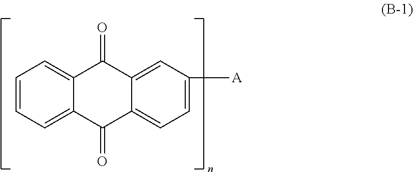

is of further interest

An anthraquinone derivative of the formula (B)

wherein n is 2 or 3,
when n is 2, A is a group —SO₂—N(H)—Z₁—N(H)—SO₂—,
Z₁ is C₂-C₂₀alkylene; and
when n is 3, A is the group

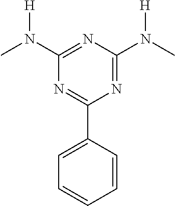

is of particular interest.

The compounds of the formulae (A) and (B) can be prepared according to or in analogy to the methods described in the working examples.

### Examples of Compounds of the Formula (A) are:

(A-1) 2-dodecyl-anthraquinone (CAS No. 71308-01-5)
(A-2) 2,6-bis[2-ethyl-hexyloxy]anthraquinone (CAS No. 332083-42-8)
(A-3) 2,6-bis[octadecyloxy]anthraquinone
(A-4) 1-(4,6-bis[dibutylamino]-[1,3,5]-triazin-2-ylamino)-anthraquinone
(A-5) 2-(4,6-bis[dibutylamino]-[1,3,5]-triazin-2-ylamino)-anthraquinone
(A-6) 2,6-bis[4,6-bis[dibutylamino]-[1,3,5]-triazin-2-yloxy]-anthraquinone
(A-7) 2-(2-ethyl-hexyloxy)-1-octadecyloxy-anthraquinone
(A-8) 1,2-bis[octadecyloxy]anthraquinone
(A-9) 1,2-bis[2-ethyl-hexyloxy]anthraquinone
(A-10) 1,2-bis[octyloxy]anthraquinone
(A-11) 1,5-bis[2-ethyl-hexyloxy]anthraquinone
(A-12) 1,5-bis[octyloxy]anthraquinone (CAS No. 180084-35-9)
(A-13) 9,10-dioxo-9,10-dihydro-anthracene-2-sulfonic acid dodecylamide
(A-14) 2,6-bis[stearoyloxy]anthraquinone

### Examples of Compounds of the Formula (B) are:

(B-1-1) bis[9,10-dioxo-9,10-dihydro-anthracene-2-sulfonic acid]-1′,12′-dodecandiyldiamide
(B-1-2) 2,6-bis[9,10-dioxo-9,10-dihydro-anthracene-2-amino]-4-phenyl-[1,3,5]-triazine (CAS No. 4118-16-5)

The major advantages of the anthraquinone derivatives of the formula (A) or (B) is their
enhanced thermostability which will ensure that no loss of active principle will occur during the processing operations,
reduced volatility which is essential in order to avoid exposure material deposition on the equipment and hazardous fumes during processing, and furthermore their
melting point, that renders them easily miscible at compounding temperatures with the matrix polymer and will lead to a homogeneous distribution of the photodegradant in the final polymer matrix, a fact that is essential to obtain homogeneous degradation throughout the final product.

An additive mixture, wherein

component (b-0) is poly-beta-pinene or 1,2-bis[2-ethylhexyloxy]anthraquinone,
component (b-I) is manganese stearate, and
component (b-II) is calcium peroxide, is particularly preferred.

Further preferred is an additive mixture comprising the components

(b-0) a polyterpene, and
(b-I) a C₂-C₃₆carboxylate of Fe, Ce, Mn, Cu or V.

Component (b-0) is preferably present in the additive mixture in an amount of 0.005 to 90% by weight, in particular 0.005 to 70% by weight, relative to the total weight of the additive mixture.

Component (b-I) is preferably present in the additive mixture in an amount of 0.005 to 90% by weight, in particular 0.005 to 70% by weight, relative to the total weight of the additive mixture.

Component (b-II) is preferably present in the additive mixture in an amount of 0.005 to 90% by weight, in particular 0.005 to 70% by weight, relative to the total weight of the additive mixture.

The additive mixture used according to the present invention may further contain one or more conventional additives. Examples are:

1. Antioxidants

1.1. Alkylated monophenols, for example 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethyl-phenol, 2,6-dioctadecyl-4-methylphenol, 2,4,6-tricyclohexylphenol, 2,6-di-tert-butyl-4-methoxymethylphenol, nonylphenols which are linear or branched in the side chains, for example 2,6-di-nonyl-4-methylphenol, 2,4-dimethyl-6-(1-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof.

1.2. Alkylthiomethylphenols, for example 2,4-dioctylthiomethyl-6-tert-butylphenol, 2,4-dioctyl-thiomethyl-6-methylphenol, 2,4-dioctylthiomethyl-6-ethylphenol, 2,6-di-dodecylthiomethyl-4-nonylphenol.

1.3. Hydroquinones and alkylated hydroquinones, for example 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate.

1.4. Tocopherols, for example α-tocopherol, β-tocopherol, γ-tocopherol, δ-tocopherol and mixtures thereof (vitamin E).

1.5. Hydroxylated thiodiphenyl ethers, for example 2, 2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)-disulfide.

1.6. Alkylidenebisphenols, for example 2, 2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butyl-phenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methyllenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane.

1.7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxy-benzyl)sulfide, isooctyl-3,5-di-tert-butyl-4-hydroxybenzylmercaptoacetate.

1.8. Hydroxybenzylated malonates, for example dioctadecyl-2,2-bis(3,5-di-tert-butyl-2-hydroxybenzyl)malonate, di-octadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol.

1.10. Triazine compounds, for example 2,4-bis(octylmercapto)-6-(3,5-di-tert-butyl-4-hydroxy-anilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)iso-cyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethyllene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylol-propane, 4-hydroxynnethyl-1-phospha-2,6,7-trioxabicyclo[2.2.2]octane.

1.14. Esters of β-(5-tert-butyl-4-hydroxy-3-methylphenyl)propionic acid with mono- or poly-hydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard® XL-1, supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetra-methyl-4,4′-diaminodiphenylmethane, 1,2-bis[(2-methylphenyl)amino]ethane, 1,2-bis(phenyl-amino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazines, N-allylphenothiazine, N,N,N′,N′-tetraphenyl-1,4-diaminobut-2-ene, N,N-bis(2,2,6,6-tetramethylpiperid-4-yl-hexamethylenediamine, bis(2,2,6,6-tetramethylpiperid-4-yl)sebacate, 2,2,6,6-tetramethylpiperidin-4-one, 2,2,6,6-tetramethylpiperidin-4-ol.

2. UV absorbers and light stabilisers

2.1. 2-(2′-Hydroxyphenyl)benzotriazoles, for example 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(α,α-dimethylbenzyl)-2′-hydroxyphenyl)benzotriazole, 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxyphenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonyl-ethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂COO—CH₂CH₂₂, where R=3′-tert-butyl-4′-hydroxy-5′-2H-benzotriazol-2-ylphenyl, 2-[2′-hydroxy-3′-(α,α-dimethyl benzyl)-5′-(1,1,3,3-tetramethylbutyl)phenyl]-benzotriazole; 2-[2′-hydroxy-3′-(1,1,3,3-tetramethylbutyl)-5′-(α,α-dimethylbenzyl)phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, for example 4-tert-butylphenyl salicylate, phenyl salicylate, octylphenyl salicylate, dibenzoyl resorcinol, bis(4-tert-butylbenzoyl)resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxycinnamate, butyl α-cyano-β-methyl-p-methoxycinnamate, methyl α-carbomethoxy-p-methoxycinnamate and N-(β-carbomethoxy-β-cyanovinyl)-2-methylindoline.

2.5. Nickel compounds, for example nickel complexes of 2,2′-thiobis[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenylundecylketoxime, nickel complexes of 1-phenyl-4-lauroyl-5-hydroxypyrazole, with or without additional ligands.

2.6. Sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-di-chloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)-malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis(2,2,6,6-tetramethyl-4-piperidyl)hexamethylene-diamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)-ethane, the condensate of 2-chloro-4,6-di-(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensate of N,N′-bis(2,2,6,6-tetramethyl-4-piperidyl)hexamethylenediamine and 4-cyclohexylamino-2,6-dichloro-1,3,5-triazine, a condensate of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); a condensate of 1,6-hexanediamine and 2,4,6-trichloro-1,3,5-triazine as well as N,N-dibutylamine and 4-butylamino-2,2,6,6-tetramethylpiperidine (CAS Reg. No. [192268-64-7]); N-(2,2,6,6-tetramethyl-4-piperidyl)-n-dodecylsuccinimide, N-(1,2,2,6,6-pentamethyl-4-piperidyl)-n-dodecylsuccin imide, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro-[4,5]decane and epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, a diester of 4-methoxymethylenemalonic acid with 1,2,2,6,6-pentamethyl-4-hydroxypiperidine, poly[methylpropyl-3-oxy-4-(2,2,6,6-tetramethyl-4-piperidyl)]siloxane, a reaction product of maleic acid anhydride-α-olefin copolymer with 2,2,6,6-tetramethyl-4-aminopiperidine or 1,2,2,6,6-pentamethyl-4-aminopiperidine.

2.7. Oxamides, for example 4, 4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2, 4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine.

3. Metal deactivators, for example N,N′-diphenyloxamide, N-salicylal-N′-salicyloyl hydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenylalkyl phosphites, phenyldialkyl phosphites, tris(nonylphenyl)phosphite, trilauryl phosphite, trioctadecyl phosphite, distearylpentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,4-di-cumylphenyl)pentaerythritol diphosphite, bis(2,6-di-tert-butyl-4-methylphenyl)pentaerythritol diphosphite, diisodecyloxypentaerythritol diphosphite, bis(2,4-di-tert-butyl-6-methylphenyl)-pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-dibenz[d,g]-1,3,2-dioxaphosphocin, bis(2,4-di-tert-butyl-6-methylphenyl)methyl phosphite, bis(2,4-di-tert-butyl-6-methylphenyl)ethyl phosphite, 6-fluoro-2,4,8,10-tetra-tert-butyl-12-methyl-dibenz[d,g]-1,3,2-dioxaphosphocin, 2,2′,2″-nitrilo-[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl)phosphite, 5-butyl-5-ethyl-2-(2,4,6-tri-tert-butylphenoxy)-1,3,2-dioxaphosphirane.

The following phosphites are especially preferred:

Tris(2,4-di-tert-butylphenyl)phosphite (Irgafos® 168, Ciba-Geigy), tris(nonylphenyl) phosphite,

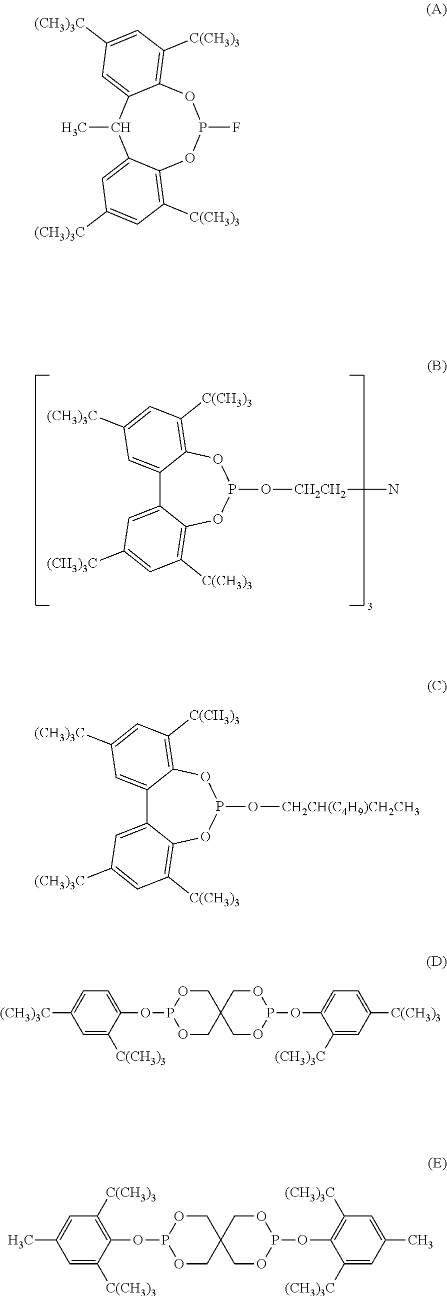

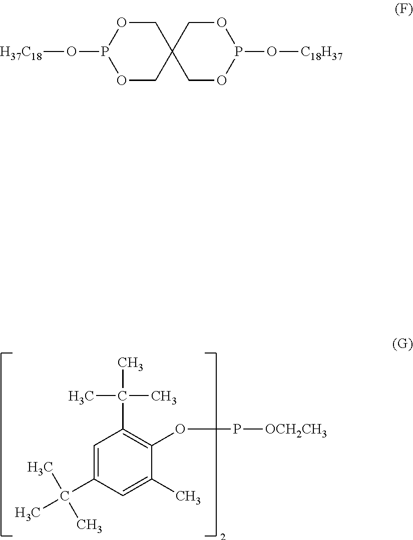

5. Hydroxylamines, for example N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxyylamine, N-heptadecyl-N-octadecylhydroxylamine, N,N-dialkylhydroxylamine derived from hydrogenated tallow amine.

6. Nitrones, for example N-benzyl-alpha-phenylnitrone, N-ethyl-alpha-methylnitrone, N-octyl-alpha-heptylnitrone, N-lauryl-alpha-undecylnitrone, N-tetradecyl-alpha-tridecylnitrone, N-hexadecyl-alpha-pentadecylnitrone, N-octadecyl-alpha-heptadecylnitrone, N-hexadecyl-alpha-heptadecylnitrone, N-ocatadecyl-alpha-pentadecylnitrone, N-heptadecyl-alpha-hepta-decylnitrone, N-octadecyl-alpha-hexadecylnitrone, nitrone derived from N,N-dialkylhydroxylamine derived from hydrogenated tallow amine.

7. Thiosvnergists, for example dilauryl thiodipropionate, or distearyl thiodipropionate.

8. Peroxide scavengers, for example esters of β-thiodipropionic acid, for example the lauryl, stearyl, myristyl or tridecyl esters, mercaptobenzimidazole or the zinc salt of 2-mercaptobenzimidazole, zinc dibutyldithiocarbamate, dioctadecyl disulfide, pentaerythritol tetrakis(β-dodecylmercapto)propionate.

9. Polyamide stabilisers, for example copper salts in combination with iodides and/or phosphorus compounds and salts of divalent manganese.

10. Basic co-stabilisers, for example melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids, for example calcium stearate, zinc stearate, magnesium behenate, magnesium stearate, sodium ricinoleate and potassium palmitate, antimony pyrocatecholate or zinc pyrocatecholate.

11. Nucleating agents, for example inorganic substances, such as talcum, metal oxides, such as titanium dioxide or magnesium oxide, phosphates, carbonates or sulfates of, preferably, alkaline earth metals; organic compounds, such as mono- or polycarboxylic acids and the salts thereof, e.g. 4-tert-butylbenzoic acid, adipic acid, diphenylacetic acid, sodium succinate or sodium benzoate; polymeric compounds, such as ionic copolymers (ionomers). Especially preferred are 1,3:2,4-bis(3′,4′-dimethylbenzylidene)sorbitol, 1,3:2,4-di(paramethyldibenzylidene)sorbitol, and 1,3:2,4-di(benzylidene)sorbitol.

12. Fillers and reinforcing agents, for example calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

13. Other additives, for example plasticisers, lubricants, emulsifiers, pigments, rheology additives, catalysts, flow-control agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839 or EP-A-0591102 or 3-[4-(2-acetoxyethoxy)-phenyl]-5,7-di-tert-butylbenzofuran-2-one, 5,7-di-tert-butyl-3-[4-(2-stearoyloxyethoxy)phenylF benzofuran-2-one, 3,3′-bis[5,7-di-tert-butyl-3-(4μ-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butylbenzofuran-2-one, 3-(3,5-dimethyl-4-pivaloyloxyphenyl)-5,7-di-tert-butylbenzofuran-2-one, 3-(3,4-dimethylphenyl)-5,7-di-tert-butylbenzofuran-2-one, 3-(2,3-dimethylphenyl)-5,7-di-tert-butylbenzofuran-2-one.

A composition which contains in addition one or more of the following components:

(b-III) a filler or reinforcing agent,
(b-IV) a pigment,
(b-V) a light stabilizer,
(b-VI) a processing additive.
(b-VII) an antioxidant,
(b-VIII) an inorganic or organic salt of Ca, Mg, Zn or Al, or an oxide of Ca, Mg, Zn or Al,
in particular
component (b-IV),
component (b-V),
is also preferred.

Examples of these components are disclosed in detail in US-A-2003-0236325 which is incorporated by reference herein.

Component (b-III) covers e.g. calcium carbonate, silicas, glass fibres, glass bulbs, talc, kaolin, mica, barium sulfate, metal oxides and hydroxides, carbon black, graphite, wood flour, flours of other natural products, synthetic fibers and metal stearates used as fillers such as calcium stearate or zinc stearate; unsaturated organic polymers such as polybutadiene, polyisoprene, polyoctenamer, or unsaturated acids such as stearic acid, oleic acid, linoleic acid or linolenic acid; and further polymers such as polyethylene oxide or polypropylene oxide.

Component (b-IV) is for example carbon black, titanium dioxide (anatase or rutile which may range in particle size from e.g. 1000 μm to 10 nm and which may optionally be surface treated) or another organic or inorganic colour pigment frequently used in agricultural applications (for example carbon black, brown, silver, red, green).

Component (b-V) is preferably a hindered amine light stabilizer (HALS) or an UV absorber. Examples of preferred hindered amine light stabilizers are also those compounds which are disclosed for example as components (A), (B) and (C) in WO-A-01/92,392 which is incorporated by reference herein and which is equivalent to U.S. patent application Ser. No. 10/257,339.

Component (b-VI) is for example an antislip/antiblock additive, a plasticizer, an optical brightener, an antistatic agent or a blowing agent.

Component (b-VII) is for example a phenolic antioxidant.

Component (b-VIII) is for example a metal stearate, e.g. calcium stearate or zinc stearate; or zinc oxide (which may range in particle size from e.g. 1000 μm to 10 nm and which may optionally be surface treated).

Component (b-III) is preferably present in the additive mixture in an amount of 0.05 to 90% by weight, in particular 0.05 to 70% by weight, relative to the total weight of the additive mixture.

Component (b-IV) is preferably present in the additive mixture in an amount of 0.05 to 90% by weight, in particular 0.5 to 70% by weight, relative to the total weight of the additive mixture.

Component (b-V) is preferably present in the additive mixture in an amount of 0.01 to 90% by weight, in particular 0.01 to 70% by weight, relative to the total weight of the additive mixture.

Component (b-VI) is preferably present in the additive mixture in an amount of 0.05 to 90% by weight, relative to the total weight of the additive mixture.

Component (b-VII) is preferably present in the additive mixture in an amount of 0.005 to 90° A) by weight, in particular 0.01 to 70% by weight, relative to the total weight of the additive mixture.

Component (b-VIII) is preferably present in the additive mixture in an amount of 0.005 to 90% by weight, in particular 0.05 to 70% by weight, relative to the total weight of the additive mixture.

Examples of component (a) are:

1. Polymers of monoolefins and diolefins, for example polypropylene, polyisobutylene, polybut-1-ene, poly-4-methylpent-1-ene, polyvinylcyclohexane, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, Vlb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).

3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, ethylene/vinylcyclohexane copolymers, ethylene/cycloolefin copolymers (e.g. ethylene/norbornene like COC), ethylene/1-olefins copolymers, where the 1-olefin is generated in-situ; propylene/butadiene copolymers, isobutylene/isoprene copolymers, ethylene/vinylcyclohexene copolymers, ethylene/alkyl acrylate copolymers, ethylene/alkyl methacrylate copolymers, ethylene/vinyl acetate copolymers, ethylene/vinyl alcohol copolymers (EVOH) or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

Homopolymers and copolymers from 1.)-4.) may have any stereostructure including syndiotactic, isotactic, hemi-isotactic or atactic; where atactic polymers are preferred. Stereoblock polymers are also included.

5. Polystyrene, poly(p-methylstyrene), poly(α-methylstyrene).

6. Aromatic homopolymers and copolymers derived from vinyl aromatic monomers including styrene, α-methylstyrene, all isomers of vinyl toluene, especially p-vinyltoluene, all isomers of ethyl styrene, propyl styrene, vinyl biphenyl, vinyl naphthalene, and vinyl anthracene, and mixtures thereof. Homopolymers and copolymers may have any stereostructure including syndiotactic, isotactic, hemi-isotactic or atactic; where atactic polymers are preferred. Stepreoblock polymers are also included.

6a. Copolymers including aforementioned vinyl aromatic monomers and comonomers selected from ethylene, propylene, dienes, nitriles, acids, maleic anhydrides, maleimides, vinyl acetate and vinyl chloride or acrylic derivatives and mixtures thereof, for example styrene/butadiene, styrene/acrylonitrile, styrene/ethylene (interpolymers), styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.

6b. Hydrogenated aromatic polymers derived from hydrogenation of polymers mentioned under 6.), especially including polycyclohexylethylene (PCHE) prepared by hydrogenating atactic polystyrene, often referred to as polyvinylcyclohexane (PVCH).

6c. Hydrogenated aromatic polymers derived from hydrogenation of polymers mentioned under 6a.).

Homopolymers and copolymers may have any stereostructure including syndiotactic, isotactic, hemi-isotactic or atactic; where atactic polymers are preferred. Stereoblock polymers are also included.

7. Graft copolymers of vinyl aromatic monomers such as styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.

8. Halogen-containing polymers such as polychloroprene, chlorinated rubbers, chlorinated and brominated copolymer of isobutylene-isoprene (halobutyl rubber), chlorinated or sulfochlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.

9. Polymers derived from α,β-unsaturated acids and derivatives thereof such as polyacrylates and polymethacrylates; polymethyl methacrylates, polyacrylamides and polyacrylonitriles, impact-modified with butyl acrylate.

10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

12. Homopolymers and copolymers of cyclic ethers such as polyalkylene glycols, polyethyllene oxide, polypropylene oxide or copolymer: thereof with bisglycidyl ethers.

13. Polyacetals such as polyoxymethylene and those polyoxymethylenes which contain ethylene oxide as a comonomer; polyacetals modified with thermoplastic polyurethanes, acrylates or MBS.

14. Polyphenylene oxides and sulfides, and mixtures of polyphenylene oxides with styrene polymers or polyamides.

15. Polyurethanes derived from hydroxyl-terminated polyethers, polyesters or polybutadienes on the one hand and aliphatic or aromatic polyisocyanates on the other, as well as precursors thereof.

16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4,-trimethylhexamethylene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).

17. Polyureas, polyimides, polyamide-imides, polyetherimids, polyesterimids, polyhydantoins and polybenzimidazoles.

18. Polyesters derived from dicarboxylic acids and diols and/or from hydroxycarboxylic acids or the corresponding lactones, for example polyethylene terephthalate, polybutylene terephthalate, poly-1,4-dimethylolcyclohexane terephthalate, polyalkylene naphthalate (PAN) and polyhydroxybenzoates, as well as block copolyether esters derived from hydroxyl-terminated polyethers; and also polyesters modified with polycarbonates or MBS.

19. Polycarbonates and polyester carbonates.

20. Polyketones.

21. Polysulfones, polyether sulfones and polyether ketones.

22. Crosslinked polymers derived from aldehydes on the one hand and phenols, ureas and melamines on the other hand, such as phenol/formaldehyde resins, urea/formaldehyde resins and melamine/formaldehyde resins.

23. Drying and non-drying alkyd resins.

24. Unsaturated polyester resins derived from copolyesters of saturated and unsaturated dicarboxylic acids with polyhydric alcohols and vinyl compounds as crosslinking agents, and also halogen-containing modifications thereof of low flammability.

25. Crosslinkable acrylic resins derived from substituted acrylates, for example epoxy acrylates, urethane acrylates or polyester acrylates.

26. Alkyd resins, polyester resins and acrylate resins crosslinked with melamine resins, urea resins, isocyanates, isocyanurates, polyisocyanates or epoxy resins.

27. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.

28. Natural polymers such as cellulose, rubber, gelatin and chemically modified homologous derivatives thereof, for example cellulose acetates, cellulose propionates and cellulose butyrates, or the cellulose ethers such as methyl cellulose; as well as rosins and their derivatives.

29. Blends of the aforementioned polymers (polyblends), for example, PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

Component (a) is preferably a thermoplastic natural or synthetic polymer.

Preferred examples of component (a) are:

a) Homo and copolymers of olefin monomers e.g. ethylene and propylene, as well as higher 1-olefins such as 1-butene, 1-pentene, 1-hexene or 1-octene. Particularly preferred is polyethylene, LDPE and LLDPE, HDPE and polypropylene.
b) Homo- and copolymers of olefin monomers with diolefin monomers such as butadiene, isoprene and cyclic olefins, e.g. norbornene.
c) Copolymers of one or more 1-olefins and/or diolefins with carbon monoxide and/or with other vinyl monomers, including but not limited to acrylic acid and its corresponding acrylic esters, methacrylic acid and its corresponding esters, vinyl acetate, vinyl ketone, styrene, maleic acid anhydride and vinyl chloride.
d) Polyvinyl alcohol.

### e) Polystyrene.

Polyolefins, in particular polyethylene, are of special interest.

According to a preferred embodiment of the present invention component (a) is a polyolefin homo- or copolymer, a starch modified polyolefin or a starch based polymer composite, in particular polyethylene, polypropylene, a polyethylene copolymer or a polypropylene copolymer.

Component (b-0) is for example present in the organic polymer (=component (a)) in an amount of 0.01 to 10%, preferably 0.01 to 5%, relative to the weight of the organic polymer.

Component (b-I) is for example present in the organic polymer (=component (a)) in an amount of 0.005 to 10%, preferably 0.005 to 5%, relative to the weight of the organic polymer.

Component (b-II) is for example present in the organic polymer (=component (a)) in an amount of 0.005 to 20% or 0.005 to 10%, preferably 0.1 to 15% or 1 to 10%, relative to the weight of the organic polymer.

Component (b-III) is preferably present in the degradable polymer in an amount of 0.05 to 80% by weight, in particular 0.5 to 70% by weight, relative to the total weight of the degradable polymer.

Component (b-IV) is preferably present in the degradable polymer in an amount of 0.05 to 40% by weight, in particular 0.5 to 30% by weight, relative to the total weight of the degradable polymer.

Component (b-V) is preferably present in the degradable polymer in an amount of 0.01 to 20% by weight, in particular 0.01 to 10% by weight, relative to the total weight of the degradable polymer.

Component (b-VI) is preferably present in the degradable polymer in an amount of 0.05 to 10% by weight, relative to the total weight of the degradable polymer.

Component (b-VII) is preferably present in the degradable polymer in an amount of 0.005 to 1% by weight, in particular 0.01 to 0.3% by weight, relative to the total weight of the degradable polymer.

Component (b-VIII) is preferably present in the degradable polymer in an amount of 0.005 to 5% by weight, in particular 0.05 to 1% by weight, relative to the total weight of the degradable polymer.

The above components can be incorporated into the thermoplastic natural or synthetic polymer or wax in a controlled form by known methods, for example before or during shaping or by applying the dissolved or dispersed compounds to the thermoplastic natural or synthetic polymer or wax, if necessary with subsequent evaporation of the solvent. The components can be added to the thermoplastic natural or synthetic polymer or wax in the form of a powder, granules or a masterbatch, which contains these components in, for example, a concentration of from 2.5 to 25% by weight.

If desired, the components (b-0) and (b-I) and optionally (b-II) to (b-VIII) can be blended with each other before incorporation into the thermoplastic natural or synthetic polymer or wax. They can be added to the polymer before or during the polymerization or before the crosslinking.

The present invention is especially useful in all areas where degradation in humid environments is desired. The use of the present additive mixture can be tailored according to the climatic conditions of the place where an article made of the above described composition will be finally stored for degradation.

A brief description of some exemplary articles made in accordance with the invention follows. Typically, the articles are required to have a relatively long service life followed by a relatively short period during which embrittlement and fragmentation occurs, either in situ or in a landfill. The articles may be film products comprising polyolefins or other organic polymers.

Examples of suitable agricultural articles are mulch films, small tunnel films, banana bags, direct covers, nonwoven, twines and pots.

In all of the foregoing examples, the article, made in accordance with the invention, will keep its properties during use and will degrade after its service life.

Mulch films represent a particular preferred embodiment of the present invention.

Mulch films are used to protect crops in the early stages of their development. Mulch films, depending on the type of crop and on the purpose, can be laid after the seeding or at the same time as the seeding. They protect the crop until the crop has reached a certain development stage. When the harvest is finished, the field is prepared for another cultivation.

Standard plastic films have to be collected and disposed in order to allow the new cultivation. The additive systems of the present invention (components (b-0) and (b-I) and optionally (b-II) to (b-VIII)), when added to the standard plastic mulch films, allow the film to keep its properties until the crop has reached the required development, then degradation starts and the film is completely embrittled when the new cultivation has to be started.

The length of the service period and of the time to degradation and time to complete disappearance depends on the type of crop and on the environmental conditions. Depending on the specific time requirements, the additive combinations are designed.

The main components of the present additive system are a prodegradant additive system (components (b-0) and (b-I) and optionally (b-II)). By appropriately dosing the amount of the prodegradant system, the required service periods and time to degradation and disappearance can be obtained. Examples of typical life times of mulch films are 10 to 180 days, lifes up to 24 months can also be required and achieved.

Thus, a further preferred embodiment of the present invention is a mulch film containing the additive mixture as defined above and having a life time of 10 to 720 days.

Mulch films can be mono or multilayer (preferably three layers), transparent or appropriately pigmented (white, black, silver, green, brown) on the base of the agronomic needs.

The thickness of the mulch films can range, for example, between 5 to 100 microns. Films from 10 to 60 microns are preferred.

A further embodiment of the present invention relates to a method for controlling the weathering resistance and the degradation of an agricultural article made of an organic polymer, which method comprises incorporating into the organic polymer the additive mixture as defined herein.

Another embodiment of the present invention is the additive mixture comprising the components

(b-0) a photosensitizer,
(b-I) an organic salt of a transition metal, and
(b-II) an inorganic peroxide or inorganic superoxide.

Further embodiments of the present invention relate to
An additive mixture comprising the components
(b-0) a photosensitizer,
(b-I) an organic salt of a transition metal, and
(b-V) a light stabilizer containing a 2,2,6,6-tetramethylpiperidin-1,4-diylgroup of the formula

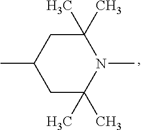

for example those listed above under item 2.6.
An additive mixture comprising the components
(b-0) a photosensitizer,
(b-I) an organic salt of a transition metal, and
(b-IV) carbon black.
An additive mixture comprising the components
(b-0) an anthraquinone derivative of the formula (A*) or (B*), and
(b-I) an organic salt of a transition metal;

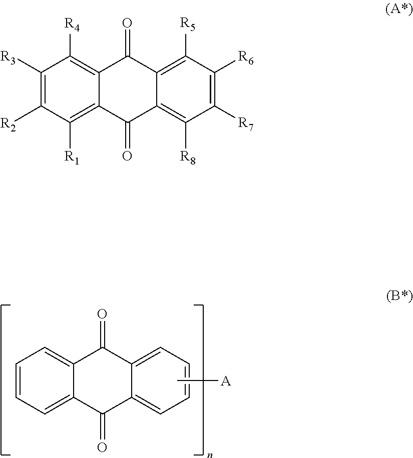

wherein R₁, R₂, R₃, R₄, R₅, R₆, R₇ and R₈ independently of one another are
hydrogen,
C₈-C₂₀alkyl,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkyloxy and hydroxy;
an unsubstituted or substituted 5 to 6 ring-membered heterocyclic group,
a polyoxyalkylene residue; or a group

### —O—X₁,

### —C(O)—X₂,

### —O—C(O)—X₃,

### —C(O)—O—X₄,

### —N(X₅)(X₆),

### —S—X₇,

### —SO₂—O—X₉, or

### —SO₂—N(X₉)(X₁₀)

X₁, X₂, X₃, X₄, X₅, X₆, X₇, X₅, X₉ and X₁₀ independently of one another are
hydrogen,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkyloxy and hydroxy;
an unsubstituted or substituted 5 to 6 ring-membered heterocyclic group, or
a polyoxyalkylene residue; and
X₁ additionally is C₈-C₂₀alkyl; and
X₂, X₃, X₄, X₅, X₆, X₇, X₈, X₉ and X₁₀ independently of one another additionally are C₁-C₂₀alkyl;
with the proviso that at least one radical of the group consisting of R₁, R₂, R₃, R₄, R₅, R₆, R₇ and R₈ is different from hydrogen;
n is 2 or 3,
when n is 2, A is a group —Y₁—Z₁—Y₂— or a group —SO₂—N(G₀)-Z₁—N(G₁)-SO₂—,
Y₁ and Y₂ independently of one another are >N-G₂, —O— or —S—,
Z₁ is C₂-C₁₂alkylene, C₂-C₁₆alkylene interrupted by oxygen, sulphur or >N-G₃; C₂-C₁₂alkenylene, C₂-C₁₂alkynylene, C₅-C₁₂cycloalkylene, C₅-C₁₂cycloalkylene-(C₁-C₄alkylene)-C₅-C₁₂cycloalkylene, C₁-C₄alkylene-(C₅-C₁₂cycloalkylene)-C₁-C₄alkylene, phenylene, phenylene-(C₁-C₄alkylene)-phenylene or C₁-C₄alkylene-phenylene-C₁-C₄alkylene or a group of the formula

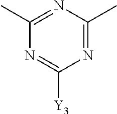

### Y₃ is —O-G₄, —S-G₅ or —N(G₆)(G₇),

G₀, G₁, G₂, G₃, G₄, G₅, G₆ and G₇ independently of one another are
hydrogen,
C₁-C₂₀alkyl,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkoxy and hydroxy;
an unsubstituted or substituted 5 to 6 ring-membered heterocyclic group, or
a polyoxyalkylene residue;
when n is 3, A is a group —Y₄—Z₂(Y₅—)(Y₆—),
Y₄, Y₅ and Y₆ independently of one another are —N(G₈)-, —O—, —S—, or —N(G₉)-SO₂—,
G₈ and G₉ independently of one another are
hydrogen,
C₁-C₂₀alkyl,
C₁-C₂₀alkyl substituted by C₁-C₁₀alkylamino, di(C₁-C₁₀alkyl)amino, C₁-C₁₀alkyloxy or hydroxy;
C₃-C₂₀alkenyl,
C₅-C₁₂cycloalkyl,
C₅-C₁₂cycloalkyl substituted by 1, 2 or 3 C₁-C₁₀alkyl;
C₅-C₉cycloalkenyl,
C₅-C₉cycloalkenyl substituted by 1, 2 or 3 C₁-C₁₀alkyl;
phenyl,
phenyl substituted by 1, 2 or 3 radicals selected from the group consisting of C₁-C₁₀alkyl,
C₁-C₁₀alkyloxy and hydroxy;
C₇-C₉phenylalkyl,
C₇-C₉phenylalkyl which is substituted on the phenyl by 1, 2 or 3 radicals selected from the group consisting of C₁-C₁₀alkyl, C₁-C₁₀alkoxy and hydroxy;
an unsubstituted or substituted 5 to 6 ring-membered heterocyclic group, or
a polyoxyalkylene residue, and
Z₂ is C₅-C₂₅alkantriyl or 2,4,6-triazintriyl.

Some anthraquinone derivatives are novel. Thus, another embodiment of the present invention relates to
2,6-bis[octadecyloxy]anthraquinone,
1-(4,6-bis[dibutylamino]-[1,3,5]-triazin-2-ylaminoyanthraquinone,
2-(4,6-bis[dibutylamino]-[1,3,5]-triazin-2-ylamino)-anthraquinone,
2,6-bis[4,6-bis[dibutylamino]-[1,3,5]-triazin-2-yloxy]-anthraquinone,
2-(2-ethyl-hexyloxy)-1-octadecyloxy-anthraquinone,
1,2-bis[octadecyloxy]anthraquinone,
1,2-bis[2-ethyl-hexyloxy]anthraquinone,
1,2-bis[octyloxy]anthraquinone,
1,5-bis[2-ethyl-hexyloxy]anthraquinone,
9,10-dioxo-9,10-dihydro-anthracene-2-sulfonic acid dodecylamide,
2,6-bis[stearoyloxy]anthraquinone, or
bis[9,10-dioxo-9,10-dihydro-anthracene-2-sulfonic acid]-1′,12′-dodecandiyldiamide.

Further embodiments of the present invention are

a composition containing
a thermoplastic natural or synthetic polymer or wax, and
at least one of the novel anthraquinone derivatives as defined above or one of the novel additive mixtures as defined above; as well as
a polymer article made of such a composition. Examples of suitable polymer articles are given herein.

The present invention also relates to

a method for controlling the weathering resistance and the degradation of a natural or synthetic polymer or wax, which comprises incorporating into the natural or synthetic polymer or wax at least one of the novel anthraquinone derivatives as defined above.

A further embodiment of the present invention is

an agricultural article made of a composition containing
(a) an organic polymer, and
(b) an anthraquinone derivative of the formula (A) or (B) as defined above.

Still further embodiments of the present invention relate to the following objects:
A polymer article made of a composition containing
(a) an organic polymer,
(b-I) 0.005-10% by weight, relative to the weight of the organic polymer, of an organic salt of a transition metal,
(b-II) 1 to 10% by weight, relative to the weight of the organic polymer, of an inorganic peroxide or inorganic superoxide.
A polymer article as defined above, wherein
component (b-I) is a C₂-C₃₆carboxylate of Fe, Ce, Co, Mn, Cu or V, and
component (b-II) is CaO₂.
A polymer article as defined above, which is an agricultural article.
A polymer article as defined above, wherein the agricultural article is selected from the group consisting of mulch films, small tunnel films, row covers, banana bags, direct covers, nonwoven, twines and pots.
A polymer article as defined above, which is a packaging material and/or which is used for consumer products.
A polymer article as defined, wherein the packaging material is for food, beverage or cosmetics.
A polymer article as defined, which is a hygienic or medical article.
A polymer article as defined above, which is selected from the group consisting of films, fibers, profiles, bottles, tanks, containers, sheets, bags, styrofoam cups, plates, blister packages, boxes, package wrappings and tapes.
A polymer article as defined above, which is shaped by injection-molding, blow-molding, compression-molding, roto-molding, slush-molding, extrusion, film casting, film blowing, calendering, thermoforming, spinning or rotational casting.
A polymer article made of a composition containing
(a) an organic polymer, and
(b-II) 1 to 10% by weight, relative to the weight of the organic polymer, of an inorganic peroxide of an alkali metal or alkaline earth metal or an inorganic superoxide of an alkali metal or zinc peroxide.
   A polymer article as defined above wherein component (b-II) is Na₂O₂, CaO₂, ZnO₂ or MgO₂.
   A polymer article as defined above wherein
   component (a) is a degradable polymer selected from the group consisting of a polyester, thermoplastic aliphatic or partially aromatic polyester urethanes, aliphatic or aliphatic-aromatic polyester carbonates and aliphatic or partially aromatic polyester amides, a polyvinyl alcohol or blends thereof; or
   blends of one or more of the afore mentioned polymers with natural or modified starch, polysaccharides, lignin, wood flour, cellulose or chitin; or
   a graft polymer.

Further examples of component (a) are

polyethylensuccinate, polybutylensuccinate, polybutylensuccinate/adipate, polybutylensuccinate/carbonate, polybutylensuccinate/terephtalate, polybutyleneadipate/terephthalate, polytetramethyleneadipate/terephthalate, polybutyleneadipate/terephthalate, polycaprolactone, poly(hydroxyalcanoates), e.g. poly 3-hydroxybutyrate, poly-3-hydroxybutyrate/octanoate copolymer, poly-3-hydroxybutyrate/hexanoate/decanoate terpolymer, and polylactic acid.

Examples of degradable polymers are cited in several articles, e.g. G. Hinrichsen et al., Macromol. Mater. Eng., 276/277, 1-24 (2000) and M. Flieger et al., Folia Microbiol. 48(1), 27-44 (2003). A comprehensive summary on degradable polyesters is given in Bioploymers, Volumes 3a, 3b and 4, of A. Steinbuechel, Y. Doi (8. eds.), Wiley VCH, Weinheim (2001).

Examples of thermoplastic aliphatic or partially aromatic polyester urethanes, aliphatic or aliphatic-aromatic polyester carbonates and aliphatic or partially aromatic polyester amides are given e.g. in U.S. Pat. No. 6,307,003. Further examples of degradable polyurethanes are given in U.S. Pat. Nos. 5,961,906, and 5,898,049. Examples of degradable polyester amides are given in U.S. Pat. No. 5,512,339. The indicated US patents are incorporated by reference herein.

The origin of the degradable polymer may either be chemical, by fermentation or by production in genetically modified plants.

The examples below illustrate the invention in greater detail. All percentages and parts are by weight, unless stated otherwise.

### EXAMPLE (A-1)

### Preparation of 2-dodecyl-anthraquinone

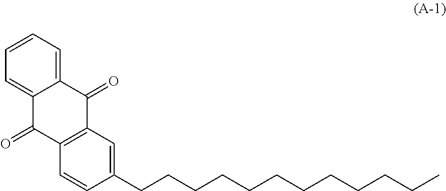

2-Dodecyl-anthraquinone is obtained in a two-step synthesis.

a) The synthesis of 2-(4-dodecyl-benzoyl)-benzoic acid is performed by reacting 45.0 g of dodecylbenzene and 29.8 g of phtalic acid anhydride in a Friedel-Crafts reaction in the presence of 500 ml of chloroform. Upon the addition of 58.5 g of AlCl₃ to the mixture of the educts, a slight exotherme is observed, followed by an evolvement of HCl gas for 1.5 hours. After the gas formation has ceased, the reaction mixture is brought to boiling until gas formation is observed again, and refluxed for another 5 hours until no more HCl gas forms. After being cooled to room temperature, the reaction mass is poured in to 300 ml of water, acidified with HCl and filtered. The crude product is washed with water until a pH of seven is reached. 2-(4-Dodecyl-benzoyl)-benzoic acid is obtained as a white crystalline solid.

Melting point: 85° C. (Yield: 48%)

NMR analysis confirms the general structure (evidence for the presence of isomers).

b) The ring closure of the 2-(4-dodecyl-benzoyl)-benzoic acid derivative is carried out by heating the compound at 90° C. for 6 hours in concentrated sulphuric acid. 2-Dodecyl-anthraquinone is obtained as a yellow solid.

Melting point: 99-105° C. (Yield: 20%)

### EXAMPLE (A-2)

### Preparation of 2,6-bis[2-ethyl-hexyloxy]anthraquinone

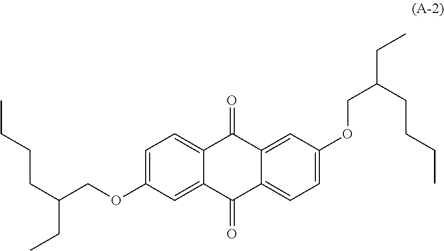

85 g of 2,6-dihydroxy-anthraquinone are added to a 2 liter round-bottom flask equipped with mechanical stirrer, thermometer and reflux condenser and containing 1000 ml of N,N′-dimethylacetamide. The mixture is heated to 80° C. until the 2,6-dihydroxy-anthraquinone is dissolved. 122 g of K₂CO₃ are added and the mixture is stirred for one hour. Then, 205.4 g of 2-ethylhexyl bromide are added dropwise within 30 minutes. Subsequently, the reaction is left at 80° C. for 8 hours. The reaction is then filtered and the solvent is evaporated by means of a rotary evaporator. The residue is taken up in toluene, washed three times with water, treated successively with active carbon, tonsil earth and filtered. After removing the solvent by evaporation, the desired product is obtained as a yellow solid which is purified by recrystallization from 400 ml of isopropanol.

Melting point: 70.9-74.7° C. (Yield: 62%)

### EXAMPLE (A-3)

### Preparation of 2,6-bis[octadecyloxy]anthraquinone

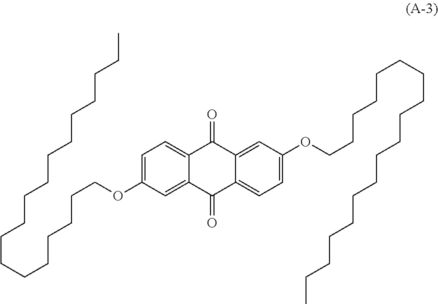

In a 500 ml round-bottom flask equipped with mechanical stirrer, thermometer, dropping funnel and condenser 10 g of 2,6-dihydroxy-anthraquinone and 16.2 g of K₂CO₃ are suspended in 100 ml of N,N′-dimethylacetamide and heated to 120° C. After one hour, 39.1 g of 1-bromo-octadecane are added dropwise. After completion of the addition, the mixture is heated for further 5 hours. After cooling to room temperature, the reaction mixture is allowed to stand overnight, during which a yellow precipitate is formed. TLC (Thin Layer Chromatography) analysis shows the presence of the starting material 2,6-dihydroxy anthraquinone, so another 5 g of K₂CO₃ are added and the reaction is re-heated for further two hours. Repeating TLC analysis confirms the complete conversion of 2,6-dihydroxy-anthraquinone. After cooling to room temperature, the mixture is filtered and a brown solid is obtained. The crude product is redissolved in toluene at 70° C. and washed three times with 100 ml of water. After evaporation of the organic solvent, the desired product is obtained as a yellow powder which is recrystallized from toluene.

Melting point: 112-122° C. (Yield: 71%)

### EXAMPLE (A-4)

### Preparation of 1-(4,6-bis[dibutylamino]-[1,3,5]-triazin-2-ylamino)-anthraquinone

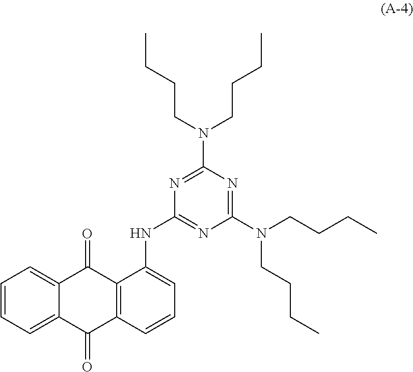

In a 500 ml round-bottom flask equipped with mechanical stirrer, thermometer and condenser 50 g of phenol are molten at 60° C. 15 g of 1-amino-anthraquinone and 49.6 g of N,N,N′,N′-tetrabutyl-6-chloro-[1,3,5]-triazine-2,4-diamine are added. The reaction is heated to 140° C. and left to react for 4 hours. The mixture is diluted with 100 ml of dichloromethane and then poured into 500 ml of water containing 26 g of NaOH to remove the phenol. The resulting precipitate is filtered, washed with 100 ml of water and 150 ml of acetone. The desired product is obtained as an orange powder without further purification.

Melting point: 128-133° C. (Yield: 94%)

### EXAMPLE (A-5)

### Preparation of 2-(4,6-bis[dibutylamino]-[1,3,5]-triazin-2-ylamino)-anthraquinone

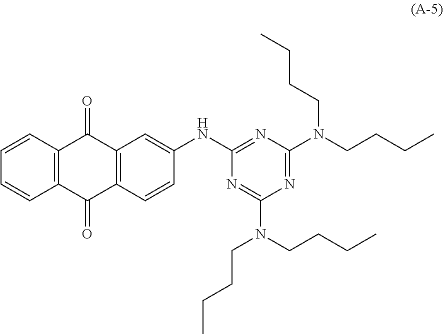

In a 500 ml round-bottom flask equipped with mechanical stirrer, thermometer and condenser 50 g of phenol are molten at 60° C. 12 g of 2-amino-anthraquinone and 38 g of N,N,N′,N′-tetrabutyl-6-chloro-[1,3,5]-triazine-2,4-diamine are added, forming a brown solution. The reaction is heated to 140° C. and left to react for 4 hours. The mixture is then poured into 50 ml of dichloromethane. This solution is added dropwise at 40° C. to 500 ml of water containing 26.5 g of NaOH. The organic volatiles are removed at 90° C. by distillation and the resulting suspension is filtered and washed with water to yield a brown powder. Recrystallization from cyclohexane gives the desired product as a yellow powder.

Melting point: 140-145° C. (Yield: 73%)

### EXAMPLE (A-6)

### Preparation of 2,6-bis[4,6-bis[dibutylamino]-[1,3,5]-triazin-2-yloxy]-anthraquinone

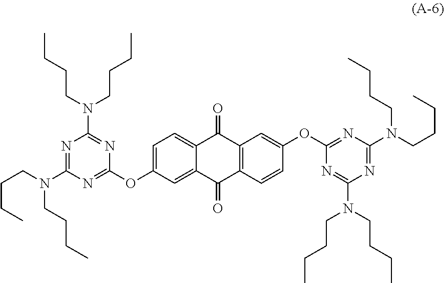

In a 500 ml round-bottom flask equipped with mechanical stirrer, thermometer, dropping funnel and condenser 300 ml of THF (tetrahydrofuran), 20 ml of water and 15 g of 2,6-dihydroxy-anthraquinone are added. The solution is cooled to 0° C. and stirred for one hour. Then, 2.75 g of NaOH are added and stirred for one hour. Subsequently, 12.65 g of cyanuric chloride dissolved in 50 ml of THF are added dropwise within 30 minutes, upon which a precipitate is formed. After completion of the addition, the reaction is stirred for another hour at 0° C. and then left to warm to 25° C. and stirred at this temperature for 12 hours. The reaction mixture is then filtered and washed with water to remove the salts. The precipitate is suspended in toluene, brought to reflux and 57 g of dibutyl amine are added dropwise within 30 minutes. The reaction is continued for eight hours at 110° C. After cooling to room temperature, the toluene phase is extracted with 200 ml of water containing 31.7 g of K₂CO₃, followed by two washings with 100 ml of water. After removal of toluene a red powder is obtained which is purified by recrystallization from isopropanol.

Melting point: 100-105° C. (Yield: 42%)

### EXAMPLE (A-7)

### Preparation of 2-(2-ethyl-hexyloxy)-1-octadecyloxy-anthraquinone

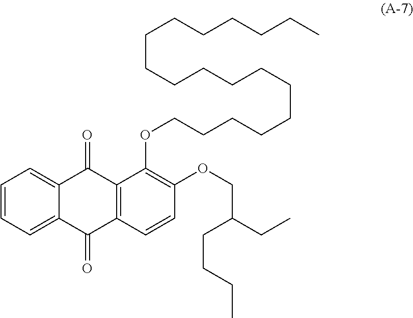

Synthesis is carried out in a 500 ml round-bottom flask equipped with mechanical stirrer, thermometer and reflux condenser. In 200 ml of N,N′-dimethylacetamide, 9.14 g of KHCO₃ and 20 g of 1,2-dihydroxy-anthraquinone (alizarin) are mixed and heated to 80° C. for one hour. 27.6 g of 2-ethylhexyl bromide are added dropwise within 30 min. After 6 hours the reaction is completed. Subsequently, 0.9 equivalent of KHCO₃ are added and the mixture is heated to 100° C. and held at this temperature. After 4 hours, 27.6 g of 1-bromo-octadecane are added to the mixture with another equivalent of KHCO₃ within 30 minutes. After two hours the temperature is increased to 125° C. for another two hours and successively 0.5 equivalent of 1-bromo-octadecane are added. The reaction is left to continue for two hours. After completion, the reaction mixture is cooled to room temperature and the precipitated product is filtered and washed with 500 ml of water. The obtained 45 g of crude product are recrystallized from isopropanol and washed with hot acetone to give 32 g of the desired compound as yellow crystals (95% HPLC (High Pressure Liquid Chromatography).

Melting point: 61-68° C. (Yield: 64%)

### EXAMPLE (A-8)

### Preparation of 1,2-bis[octadecyloxy]anthraquinone

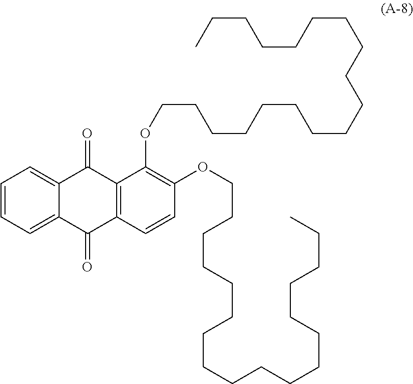

20 g of 1,2-dihydroxy-anthraquinone and 34.4 g of K₂CO₃ are added in a 500 ml round-bottom flask equipped with mechanical stirrer, thermometer and reflux condenser containing 250 ml N,N-dimethylacetamide. The mixture is heated to 80° C. and subsequently stirred for 60 minutes. Then, 82.9 g of 1-bromo-octadecane are added portionwise within one hour. The reaction is left to continue for 6 hours. After cooling to ambient temperature, a dense precipitate is formed. 100 ml of additional solvent are added to render the mixture filterable. The filter cake is washed with 50 ml of N,N-dimethylacetamide and 200 ml of water. The residue is taken up again in N,N-dimethylacetamide, filtered and washed with 200 ml of water. The desired product is obtained as a yellow powder.

Melting point: 100-107° C. (Yield: 93%)

### EXAMPLE (A-9)

### Preparation of 1,2-bis[2-ethyl-hexyloxy]anthraquinone

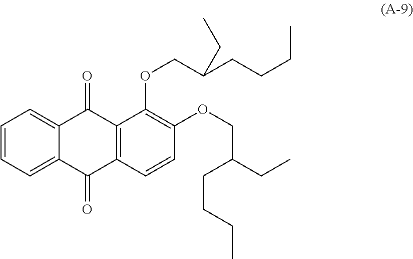

100 g of 1,2-dihydroxy-anthraquinone are added in a 2 litre round-bottom flask equipped with mechanical stirrer, thermometer and reflux condenser containing 500 ml of N,N-dimethylacetamide. The mixture is heated to 80° C. for 30 minutes. 172.6 g of K₂CO₃ are added and the mixture is left at 80° C. for one hour. Subsequently, 241.8 g of 2-ethylhexyl bromide are added dropwise within one hour. The reaction is left to react for a total of eight hours. Then, another 0.5 equivalent of K₂CO₃ are added and the heating is continued for another hour at 80° C. to complete the reaction. After filtration to remove the salts, the solvent is evaporated. The residual brown oil is dissolved in 500 ml of toluene, treated with active carbon and tonsil earth, washed three times with 200 ml of water and finally dried over sodium sulfate. 146 g of the desired product are obtained as an orange oil.

No melting point (liquid)

(Yield: 75%)

### EXAMPLE (A-10)

### Preparation of 1,2-bis[octyloxy]anthraquinone

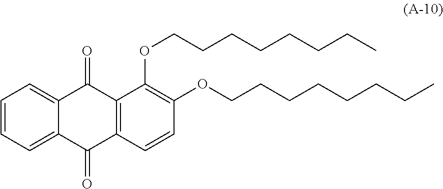

A four-necked round-bottom flask equipped with a mechanical stirrer, thermocouple, dropping funnel and condenser is charged successively with 250 ml of N,N-dimethylacetamide, 50 g of 1,2-dihydroxy-anthraquinone and 86.3 g of K₂CO₃. The mixture is heated to 80° C. and stirred at this temperature for one hour. 100.9 g of 1-octylbromide are added dropwise within 30 minutes. The reaction is allowed to proceed at 80° C. for 4 hours. Upon cooling to room temperature, the product precipitates from the solution to form a dense pulp. 50 ml of N,N-dimethylacetamide are added to improve the handling of the mixture. The reaction mass is then filtered, washed with 50 ml of N,N-dimethylacetamide and finally with water to remove the salts formed. The remaining solid is dissolved in 500 ml of CH₂Cl₂, washed three times with 200 ml of water, dried with anhydrous Na₂SO₄ and treated with tonsil earth and active carbon. After filtration over tonsil earth, the resulting yellow solution is concentrated by means of a rotary evaporator to yield 70 g of the desired product as a yellow solid.

Melting point: 91-94° C. (Yield: 72%)

### EXAMPLE (A-11)

### Preparation of 1,5-bis[2-ethyl-hexyloxy]anthraquinone

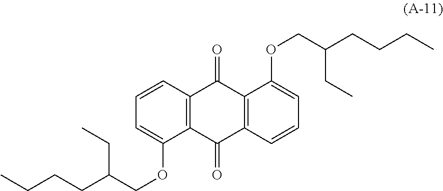

A 2 liter autoclave is charged with 750 ml of water and 41 g of KOH. Then, 50 g of 1,5-dihydroxyanthraquinone are added under slow stirring, subsequently 120 g of 2-ethylhexyl bromide and 0.5 g of benzyl-dimethyl-octyl-ammonium chloride (phase transfer catalyst) are added. The stirring frequency is increased to 250 rpm and the reaction mass is heated to 170° C. within the course of one hour. After reaching this temperature, the reaction is continued for 8 hours. Then, the mixture is cooled to 80° C. and the reaction mass is poured into a beaker and allowed to cool to room temperature. Under stirring, 800 ml of CH₂Cl₂ are added and the mixture is stirred for 30 minutes. The resulting two phases are separated by means of a separation funnel. The organic phase is washed three times with water and successively treated with tonsil earth and active carbon. The solvent is then evaporated with a rotary evaporator. The obtained dark residue is recrystallized twice from isopropanol (solvent to solid ratio 2:1 volume/weight). GLC (Gas Liquid Chromatography) analysis reveals 84% of di- and 14% of mono-substituted product. After a third crystallization step from isopropanol, 21.2 g of a yellow solid are obtained. HPLC (High Pressure Liquid Chromatography) and ¹H-NMR analysis confirms the removal of the mono-substituted derivative.

Melting point: 105° C. (Yield: 22%)

### EXAMPLE (A-12)

### Preparation of 1,5-bis[octyloxy]anthraquinone

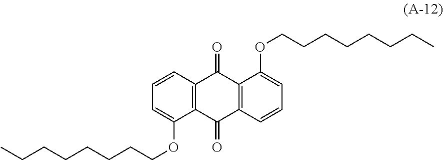

The general synthesis procedure as described in Example (1-b-1-13) is applied. The reaction is terminated after 8 hours. The aqueous phase is taken up in 2×500 ml of CH₂Cl₂ and separated. The organic phase is then washed three times with 200 ml of water. After treatment with tonsil earth, active carbon and anhydrous Na₂SO₄, the resulting orange solution is concentrated by means of a rotary evaporator. The obtained brown solid is recrystallized twice from isopropanol (2:1) to yield an orange solid. HPLC analysis confirms the presence of 22% of mono-substituted product. Consequently, the solid is redissolved in 250 ml of CH₂Cl₂ and the solution is treated with 100 ml of a 25 wt % aqueous solution of KOH. The formed precipitate is filtered off and the resulting solution is concentrated to yield an orange-yellow solid, which is recrystallized from isopropanol. 34.2 g of a yellow solid is obtained. HPLC and ¹H-NMR analysis confirms the removal of the mono-substituted derivative.

Melting point: 90-94° C. (Yield: 35%)

### EXAMPLE (A-13)

### Preparation of 9,10-dioxo-9,10-dihydro-anthracene-2-sulfonic acid dodecylamide

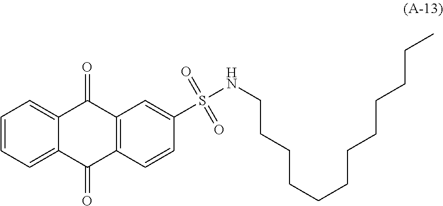

A 1 liter round bottom flask equipped with mechanical stirrer, thermocouple, dropping funnel and condenser is charged with 30 g of 9,10-dioxo-9,10-dihydro-anthracene-2-sulfonyl chloride suspended in 300 ml of THF. Then, 37.17 g of 1-dodecylamine are dissolved in 150 ml of THF and are added dropwise within 15 minutes at room temperature. A temperature increase of 20° C. is observed while adding the amine. The solution is brought to reflux for 4 hours and then recooled to room temperature, accompanied by a partial precipitation of the final product from the solution. 300 ml of water are added, leading to the precipitation of the desired product. After filtration, the precipitate is washed 4 times with 300 ml of water and 42.5 g of a white powder are obtained.

Melting point: 157-165° C. (Yield: 95%)

### EXAMPLE (A-14)

### Preparation of 2,6-bis-stearoyloxy-anthraquinone

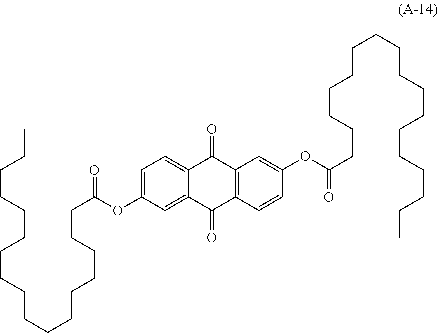

A 4 necked nitrogen filled 100 ml round-bottom flask equipped with a magnet as stirrer, thermocouple, dropping funnel and reflux condenser is charged at room temperature with 30 ml THF, 2.4 g of 2,6-dihydroxyanthraquinone and 15 ml of triethylamine. Then, 18.1 g of stearoyl chloride are added at room temperature. The solution is maintained at 30° C. for 24 hours and concentrated in vacuo with a rotary evaporator. The resulting residue is washed with THF and water and dried. 3.1 g of the desired product are obtained as a grew solid.

Melting point: 129-132° C. (Yield: 40%)

### EXAMPLE (B-1-1)

### Preparation of bis[9,10-dioxo-9,10-dihydro-anthracene-2-sulfonic acid]-1′,12′-dodecandiyldiamide

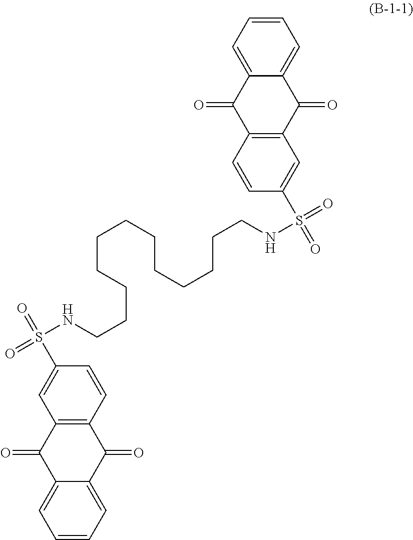

The dimeric structure is obtained using 2 equivalents of 9,10-dioxo-9,10-dihydro-anthracene-2-sulfonyl chloride and reacting it with 1 equivalent of 1,12 dodecandiyl diamine. In a 4-necked 100 ml round bottom flask equipped with mechanical stirrer, thermocouple and condenser, 1.5 g of 9,10-dioxo-9,10-dihydro-anthracene-2-sulfonyl chloride are dissolved in 30 ml of THF and stirred for 10 min. Then, 0.49 g of the diamine are added in small portions. After 15 min., 10 ml of an aqueous solution containing 0.74 g of K₂CO₃ are added dropwise. The solution rapidly thickens and is heated to 60-65° C. for 2 hours. After cooling, the resulting precipitate is filtered, washed with water and dried. 1.7 g of the desired product are obtained as a white solid.

Melting point: 238° C. (Yield: 95%)

### EXAMPLE A

### 1) Preparation of a 50 μm Thick Film

In a turbo mixer (Caccia, Labo 10) 1% of the additive indicated in the table is mixed with linear low density polyethylene (LLDPE; Dowlex NG 5056-E®; melt index: 1.1 g/10 min. at 190° C. and 2.16 Kg). The LLDPE contains 0.12% of tris(2,4-di-t-butylphenyl) phosphite, 0.02% of pentaerythritol tetrakis[3-(3,5-di-tert-butyl-4-hydroxyphenyl)propionate, and 0.03% of octadecyl-3-(3,5-di-tert-butyl-4-hydroxyphenyl)propionate. The mixture is extruded to granules at a maximum temperature of 200° C., using an O.M.C. twin-screw extruder (model ebv 19/25). The granules are subsequently converted to a 50 μm thick film, using a blow-extruder (Formac) working at a maximum temperature of 210° C.

### 2) Preparation of 25 μm Thick Films

The procedure described above under 1) is repeated except that 10% of the additive are used, and the obtained polymer granules are subsequently diluted 1 to 10 w/w with the same LLDPE and converted to a 25 μm thick film, using a blow-extruder (Dolci®) working at a maximum temperature of 210° C.

(“%” means “% by weight” relative the LLDPE.)

### 3) Exposure

a) Film samples are exposed in an ATLAS Weatherometer (WOM; model Ci65A) equipped with a 6500W Xenon lamp (continuous light cycle, black panel temperature=63° C.).

b) Films are exposed outdoor part over and part under gardening soil as in the typical mulch film setup. Exposure is carried out from June to September in Pontecchio Marconi (Bologna—Italy) where the average irradiation is about 110 KLys/year.

### Evaluation Parameters:

Carbonyl increment (CO): Evaluation of the carbonyl band increment (1710 cm-1) in function of the exposure time is monitored with a FT-IR Perkin-Elmer Spectrum One. (High values indicate the degradation of the polymer.)

Time to cracking: Visual failure of film samples is assessed according to “time to the first evidence of surface cracking”. In outdoor exposure the “time to cracking” is evaluated on the part exposed on top of the soil.

The following tables illustrate the growth of carbonyl increment measured via IR, of polyethylene films in WOM exposure: Reference material is a film containing no additive.

TABLE A-1
WOM exposure (in hours) of 50 μm LLDPE films
containing 1% of the indicated additive

| | Carbonyl growth | | | | |
|---|---|---|---|---|---|
| Hours | (None) | Compound (A-4) | Compound (A-8) | Compound (A-6) | Compound (A-9) |
| 187 | 0.013 | 0.380 | 0.170 | 0.185 | 0.200 |
| 416 | 0.048 | 0.562 | 0.330 | 0.318 | 0.340 |
| 585 | 0.091 | 0.643 | 0.440 | 0.395 | 0.452 |
| 812 | 0.185 | 0.775 | 0.630 | 0.560 | 0.628 |
| 1040 | 0.329 | 0.959 | 0.849 | | |

TABLE A-2
WOM exposure (in hours) of 50 μm LLDPE
films containing 1% of the indicated additive

| | Carbonyl growth | | |
|---|---|---|---|
| | | Compound | Compound |
| Hours | (None) | (A-3) | (A-5) |
| 213 | 0.028 | 0.338 | 0.514 |
| 428 | 0.106 | 0.537 | 0.672 |
| 623 | 0.195 | 0.665 | |
| 890 | 0.423 | 0.950 | |
| 1095 | 0.649 | 1.180 | |

TABLE A-3
WOM exposure of 50 μm LLDPE films
containing 1% of the indicated additive

| | | Compound | Compound |
|---|---|---|---|
| | (None) | (A-3) | (A-5) |
| Hours to cracking | 2647 | 1190 | 890 |

TABLE A-4
WOM exposure (in hours) of 50 μm LLDPE
films containing 1% of the indicated additive

Carbonyl growth

| | | Compound |
|---|---|---|
| Hours | (None) | (A-13) |
| 236 | 0.035 | 0.329 |
| 412 | 0.101 | 0.491 |
| 655 | 0.236 | 0.699 |
| 884 | 0.400 | 0.891 |

TABLE A-5
WOM exposure (in hours) of 25 μm LLDPE
films containing 1% of the indicated additive

| | Carbonyl growth | | | |
|---|---|---|---|---|
| | | Compound | Compound | Compound |
| Hours | (None) | (A-2) | (A-4) | (A-9) |
| 481 | 0.050 | 0.307 | 0.260 | 0.236 |
| 663 | 0.086 | 0.387 | 0.313 | 0.306 |
| 900 | 0.157 | 0.517 | 0.408 | 0.418 |

TABLE A-6
WOM exposure of 25 μm LLDPE films
containing 1% of the indicated additive

| | | Compound | Compound | Compound |
|---|---|---|---|---|
| | (None) | (A-2) | (A-4) | (A-9) |
| Hours to cracking | 1025 | 622 | 596 | 717 |

TABLE A-7
Outdoor exposure of 25 μm PE films
containing 1% of the indicated additive

| | | Compound | Compound | Compound |
|---|---|---|---|---|
| | (None) | (A-2) | (A-9) | (A-4) |
| KLys to cracking | 50 | 25 | 25 | 20 |

TABLE A-8
WOM exposure (in hours) of 50 μm LLDPE
films containing 1% of the indicated additive

Carbonyl growth

| | | Compound |
|---|---|---|
| Hours | (None) | (A-7) |
| 215 | 0.012 | 0.179 |
| 402 | 0.045 | 0.299 |
| 611 | 0.095 | 0.438 |
| 823 | 0.178 | 0.581 |
| 1078 | 0.305 | 0.770 |
| 1281 | 0.433 | |

TABLE A-9
WOM exposure of 50 μm LLDPE films
containing 1% of the indicated additive

| | | Compound |
|---|---|---|
| | (None) | (A-7) |
| Hours to cracking | 1634 | 941 |

TABLE A-10
WOM exposure (in hours) of 25 μm LLDPE
films containing 1% of the indicated additive

Carbonyl growth

| | | Compound |
|---|---|---|
| Hours | (None) | (A-10) |
| 221 | 0.011 | 0.148 |
| 478 | 0.067 | 0.258 |
| 653 | 0.119 | 0.348 |
| 843 | 0.200 | 0.431 |
| 1075 | 0.321 | |

TABLE A-11
WOM exposure of 25 μm LLDPE films
containing 1% of the indicated additive

| | | Compound |
|---|---|---|
| | (None) | (A-10) |
| Hours to cracking | 913 | 603 |

TABLE A-12
WOM exposure (in hours) of 50 μm LLDPE
films containing 1% of the indicated additive

| | Carbonyl growth | | |
|---|---|---|---|
| | | Compound | Compound |
| Hours | (None) | (A-11) | (A-12) |
| 212 | 0.024 | 0.284 | 0.314 |
| 400 | 0.089 | 0.489 | 0.528 |
| 637 | 0.217 | 0.698 | 0.733 |
| 894 | 0.396 | 0.937 | 1.006 |
| 1112 | 0.570 | | |

TABLE A-13
WOM exposure of 50 μm LLDPE films
containing 1% of the indicated additive

| | | Compound | Compound |
|---|---|---|---|
| | (None) | (A-11) | (A-12) |
| Hours to cracking | 989 | 754 | 894 |

### EXAMPLE B

### (1) Preparation of Films

Master batches of additives are obtained according to the following procedure:

Additives are mixed with linear low density polyethylene (LLDPE; Dowlex NG 5056-E®; melt index: 1.1 g/10 min. at 190° C. and 2.16 Kg). The LLDPE contains 0.12% of tris(2,4-di-t-butylphenyl) phosphite, 0.02% of pentaerythritol tetrakis[3-(3,5-di-tert-butyl-4-hydroxyphenyl)propionate, and 0.03% of octadecyl-3-(3,5-di-tert-butyl-4-hydroxyphenyl)propionate.

Further additives as indicated in the table below are:

Manganese stearate (from Shepherd Chemical Company)
Calcium peroxide (Ixper 75C® from Solvay-Interox)
Poly-β-pinene (Dercolyte S115® from DRT)

Powder of LLDPE and additives are homogenised either in a drum hoop mixer (Rhönrad) or in a turbo mixer (Caccia, Labo 10). The mixture is extruded to granules at a maximum temperature of 200° C., using either an O.M.C. twin-screw extruder (model ebv 19/25) or a Comac twin-screw extruder.

The granules are subsequently diluted to the specified loading with the same LLDPE and converted to a 25 μm thick film, using a blow-extruder (Dolci®) working at a maximum temperature of 210° C.).

| | Film thickness | |
|---|---|---|
| Ref. | in μm | Further Additives |
| Film 1 | 25 | None |
| Film 2 | 25 | 0.14% of Manganese stearate |
| | | 5% of Calcium peroxide |
| | | 1% of Poly-β-pinene (Dercolyte S115 |
| | | (RTM) - DRT) |

(“%” means “% by weight” relative the LLDPE.)

### (2) Exposures

Films are exposed outdoor part over and part under gardening soil as in the typical mulch film setup. Exposure is carried out starting early summer in Pontecchio Marconi (Bologna—Italy) where the average irradiation is about 110 KLys/year.

### (3) Evaluation Parameters

a) Carbonyl increment (CO): Evaluation of the carbonyl absorbance band increment (1710 cm-1) in function of the exposure time is monitored with a FT-IR Perkin-Elmer Spectrum One. (High values indicate the degradation of the polymer.)

b) Visual appearance of films: Visual failure (cracking) of film samples is assessed according to “time to the first evidence of surface cracking”. Visual cracking or disappearance of film samples exposed outdoor is assessed both on the part exposed on top of the soil and under the soil.

The results are indicated in the following table.

TABLE B-1
Outdoor exposure of 25 μm LLDPE films topsoil

| KLys | Film 1 | Film 2 |
|---|---|---|
| to cracking | >29.4 | 12.5 |
| to disappearance | >29.4 | 37.7 |

### EXAMPLE C

An additive masterbatch comprising cobalt stearate and 1.35 weight % of calcium carbonate and CaO₂ (obtainable from Aldrich®) is mixed with LLDPE (Dowlex® 5056NG) having a melt index of 1.1 g/10 min (190° C./2.16 Kg) in a turbo mixer (Caccia®, Labo 10). The mixture is extruded at a maximum temperature of 200° C. using an O.M.C. extruder (model ebv 19/25) to granules, which are subsequently converted to a film 50 μm thick, using a blow-extruder (Formac®) working at a maximum temperature of 210° C.

The film samples obtained are listed in Table C-1.

TABLE C-1

| | Component (I) | Component (II) |
|---|---|---|
| Film Sample | (Metal component) | (Oxidant) |
| A | — | — |
| B | 0.13 weight % of Co stearate | — |
| C | 0.13 weight % of Co stearate | 2.5 weight % of CaO₂ |
| D | 0.13 weight % of Co stearate | 5.0 weight % of CaO₂ |

### Evaluation Parameters:

Time to cracking: Visual failure of film samples is assessed according to time to the first evidence of surface cracking.
Carbonyl increment (CO): Evaluation of the carbonyl band increment (1710 cm⁻¹) in function of the exposure time is monitored with a FT-IR Perkin-Elmer Spectrum One. The carbonyl increment is a widespread criteria for the analysis and evaluation of polyolefin degradation.

### Test Methods:

1) Oven Ageing at 50° C. with Films Immersed in Water:

Film samples B to D, enclosed in quartz cells filled with water (Carlo Erba, grade Water plus for HPLC), are exposed in a circulating air oven (Heraeus®, model UT 6120). The development of the carbonyl increment dependent on the exposure time is shown in Table C-2. The time to cracking is listed in Table C-3.

TABLE C-2

| | Exposure time | |
|---|---|---|
| Sample | in hours | Carbonyl increment |
| B | 980 | 0.0 |
| C | 980 | 0.2 |
| D | 980 | 0.6 |

The results clearly demonstrate the accelerated degradation in the presence of CaO₂ (samples C and D).

TABLE C-3

| Film sample | B | D |
|---|---|---|
| Hours to cracking | >3300 | 1385 |

Not only the carbonyl increment but also the time to cracking confirms the accelerated degradation in the presence of CaO₂. Film B shows no signs of cracking, while Film D containing 5 weight % of CaO₂ cracked in less than half the exposure time.

### 2) Exposure Time in Oven at 70° C. and 100% Humidity:

In this case, the films are not completely immersed in water but the quartz flask is only filled at the bottom with water and exposed in the oven (Horo®, model 080V). The development of the carbonyl increment dependent on the exposure time is shown in Table C-4. The time to cracking is listed in Table C-5.

TABLE C-4

| | Exposure time | |
|---|---|---|
| Sample | in hours | Carbonyl increment |
| A | 215 | 0.0 |
| B | 215 | 0.0 |
| C | 215 | 0.6 |
| D | 215 | 0.6 |

TABLE C-5

| Film sample | A | B | C | D |
|---|---|---|---|---|
| Hours to cracking | >381 | >381 | 215 | 215 |

Changing the reaction set-up in order to allow an increased availability of oxygen in order to facilitate the oxidation of the film confirms the results shown in Tables C-2 and C-3, however on a much shorter time scale.

### 3) Exposure in Earth at 80° C.:

Conventional humid garden earth (obtained in a garden center) is filled into a plastic box with lid. Films of materials A, B, C and D are fixed in frames for diapositives and stuck into the earth. The lid is closed and the samples are put into a lab oven at 80° C. Films are controlled weekly regarding their visual appearance. After 5 weeks, Film D turns brownish and disappears. Inspecting the earth, no pieces of plastic can be detected visually. Film C shows strong yellowing and severe structural damages (holes and parts of film missing). After 5 weeks, Film B shows a slight yellowing but no cracks. Film A is still intact and exhibits neither signs of yellowing nor of structural damage.

1. An anthraquinone compound selected from the group consisting of
2,6-bis[octadecyloxy]anthraquinone,
1-(4,6-bis[dibutylamino]-[1,3,5]-triazin-2-ylamino)-anthraquinone,
2-(4,6-bis[dibutylamino]-[1,3,5]-triazin-2-ylaminoyanthraquinone,
2,6-bis[4,6-bis[dibutylamino]-[1,3,5]-triazin-2-yloxy]-anthraquinone,
2-(2-ethyl-hexyloxy)-1-octadecyloxy-anthraquinone,
1,2-bis[octadecyloxy]anthraquinone,
1,2-bis[2-ethyl-hexyloxy]anthraquinone,
1,2-bis[octyloxy]anthraquinone,
1,5-bis[2-ethyl-hexyloxy]anthraquinone,
9,10-dioxo-9,10-dihydro-anthracene-2-sulfonic acid dodecylamide,
2,6-bis[stearoyloxy]anthraquinone and
bis[9,10-dioxo-9,10-dihydro-anthracene-2-sulfonic acid]-1′,12′-dodecandiyldiamide.

2. A composition containing
a thermoplastic natural or synthetic polymer or a wax, and
at least one anthraquinone compound selected from the group consisting of
2,6-bis[octadecyloxy]anthraquinone,
1-(4,6-bis[dibutylamino]-[1,3,5]-triazin-2-ylamino)-anthraquinone,
2-(4,6-bis[dibutylamino]-[1,3,5]-triazin-2-ylamino)-anthraquinone,
2,6-bis[4,6-bis[dibutylamino]-[1,3,5]-triazin-2-yloxy]-anthraquinone,
2-(2-ethyl-hexyloxy)-1-octadecyloxy-anthraquinone,
1,2-bis[octadecyloxy]anthraquinone,
1,2-bis[2-ethyl-hexyloxy]anthraquinone,
1,2-bis[octyloxy]anthraquinone,
1,5-bis[2-ethyl-hexyloxy]anthraquinone,
9,10-dioxo-9,10-dihydro-anthracene-2-sulfonic acid dodecylamide,
2,6-bis[stearoyloxy]anthraquinone and
bis[9,10-dioxo-9,10-dihydro-anthracene-2-sulfonic acid]-1′,12′-dodecandiyldiamide.

3. An agricultural article made of a composition containing
(a) an organic polymer and
(b) an additive mixture comprising
(b-0) 2,6-bis[octadecyloxy]anthraquinone,
1-(4,6-bis[dibutylamino]-[1,3,5]-triazin-2-ylamino)-anthraquinone,
2-(4,6-bis[dibutylamino]-[1,3,5]-triazin-2-ylaminoyanthraquinone,
2,6-bis[4,6-bis[dibutylamino]-[1,3,5]-triazin-2-yloxy]-anthraquinone,
2-(2-ethyl-hexyloxy)-1-octadecyloxy-anthraquinone,
1,2-bis[octadecyloxy]anthraquinone,
1,2-bis[2-ethylhexyloxy]anthraquinone,
1,2-bis[octyloxy]anthraquinone,
1,5-bis[2-ethyl-hexyloxy]anthraquinone,
9,10-dioxo-9,10-dihydro-anthracene-2-sulfonic acid dodecylamide,
2,6-bis[stearoyloxy]anthraquinone or
bis[9,10-dioxo-9,10-dihydro-anthracene-2-sulfonic acid]-1′,12′-dodecandiyldiamide and
(b-I) a C₂-C₃₆carboxylate of Fe, Ce, Co, Mn, Cu or V.

4. A agricultural article according to claim 3 further comprising
(b-II) an inorganic peroxide of an alkali metal, an alkaline earth metal or a transition metal, or an inorganic superoxide of an alkali metal, an alkaline earth metal or a transition metal.

5. A agricultural article according to claim 4 where the peroxide or superoxide is magnesium peroxide, calcium peroxide, strontium peroxide, barium peroxide, lithium peroxide, sodium peroxide, potassium peroxide, zinc peroxide, silver peroxide, copper peroxide, iron peroxide, lithium superoxide, sodium superoxide, potassium superoxide, rubidium superoxide or cesium superoxide.

6. An agricultural article according to claim 4 where
component (b-0) is 1,2-bis[2-ethylhexyloxy]anthraquinone,
component (b-I) is manganese stearate and
component (b-II) is calcium peroxide.

7. An agricultural article according to claim 3, further comprising one or more components selected from
(b-III) a filler or reinforcing agent,
(b-IV) a pigment,
(b-V) a light stabilizer,
(b-VI) a processing additive,
(b-VII) an antioxidant and
(b-VIII) an inorganic or organic salt of Ca, Mg, Zn or Al, or an oxide of Ca, Mg, Zn or Al.

8. An agricultural article according to claim 3, further comprising one or more components selected from
(b-IV) a pigment and
(b-V) a light stabilizer.

9. An agricultural article according to claim 3 wherein
component (a) is a thermoplastic natural or synthetic polymer.

10. An agricultural article according to claim 3 wherein
component (a) is a polyolefin homo- or copolymer, a starch modified polyolefin or a starch based polymer composite.

11. An agricultural article according to claim 3 wherein
component (a) is polyethylene, polypropylene, a polyethylene copolymer or a polypropylene copolymer.

12. An agricultural article according to claim 3 which is selected from the group consisting of mulch films, small tunnel films, banana bags, direct covers, nonwoven, twines and pots.

13. An agricultural article according to claim 3 which is a mulch film.

14. A method for controlling the weathering resistance and the degradation of an agricultural article made of an organic polymer, which method comprises incorporating into the organic polymer an additive mixture as defined in claim 3.

15. An additive mixture comprising the components
(b-0) as photosensitizer, an anthraquinone compound according to claim 1,
(b-I) an organic salt of a transition metal and
(b-II) an inorganic peroxide or inorganic superoxide.

16. An additive mixture comprising the components
(b-0) as photosensitizer, an anthraquinone compound according to claim 1,
(b-I) an organic salt of a transition metal and
(b-V) a light stabilizer containing a 2,2,6,6-tetramethylpiperidin-1,4-diyl group of the formula

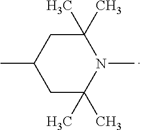

17. An additive mixture comprising the components
(b-0) as photosensitizer, an anthraquinone compound according to claim 1,
(b-I) an organic salt of a transition metal and
(b-IV) carbon black.
